(19) United States
(12) Patent Application Publication
(10) Pub. No.: US 2025/0108045 A1
(43) Pub. Date: Apr. 3, 2025

(54) Diagnostic Opioid Taper Regimen

(71) Applicant: Joseph Leon Grossman, Boulder, CO (US)

(72) Inventor: Joseph Leon Grossman, Boulder, CO (US)

(21) Appl. No.: 18/375,817

(22) Filed: Oct. 2, 2023

(51) Int. Cl. A61K 31/485; A61K 9/00; A61K 9/20

(52) U.S. Cl. CPC A61K 31/485; A61K 9/006; A61K 9/2095

(57) ABSTRACT

The invention, the Diagnostic Opioid Taper Regimen, consists of a sequence of dosages of buprenorphine. There are a series of dosage levels in the sequence. Each new dosage level is administered for a period of time, such as several weeks. The rate of dosage reduction is increased each time the dosage is reduced. This increasing rate of dosage reduction exposes the patient to a sequence of increasingly rapid dosage reductions. When the rate of dosage reduction exceeds the rate at which the patient is comfortable, the patient will start to experience symptoms of withdrawal.

The emergence of symptoms of withdrawal indicates the maximum rate of dosage reduction at which the patient is comfortable tapering has been exceeded. The provider uses this information to determine an appropriate rate of dosage reduction for that patient. The diagnostic taper is designed to be utilized with various medications that can cause dependency.

### CROSS-REFERENCE TO RELATED APPLICATIONS

This application claims the benefit of priority of U.S. Provisional Patent Application No. 63/437,009 filed Jan. 4, 2023, titled Diagnostic Taper, and also U.S. Pat. No. 11,253,512,B2, issued Feb. 22, 2022, titled Opioid Taper Regimen, both hereby incorporated by reference.

### STATEMENT RE: FEDERALLY SPONSORED RESEARCH OR DEVELOPMENT

No prior or current federally sponsored research or development.

### NAMES OF THE PARTIES TO A JOINT RESEARCH AGREEMENT

Not applicable

### REFERENCE TO A “SEQUENCE LISTING”

Not applicable

### PRIOR DISCLOSURES BY THE INVENTOR OR A JOINT INVENTOR

None.

### BACKGROUND OF THE INVENTION

The invention is in the field of addiction medicine. The invention, which is a diagnostic regimen of medication, advances the ability to evaluate and treat patients diagnosed with substance use disorders and substance dependency, such as opioid dependency. There are approximately 2.4 million persons in the U.S. diagnosed with opioid use disorder.

The treatment of opioid use disorder and related conditions is the focus of many public health programs. Approximately 670,000 patients are in opioid maintenance programs. These programs generally aim to address opioid dependency and improve quality of life by combining the administration of medication for opioid use disorders with counseling and support services. “Administration” here means the prescribing of medication that is then consumed by patients as intended. The medications most used in opioid maintenance programs are buprenorphine and methadone. This invention is intended to serve patients in opioid maintenance programs who want to reduce or stop their opioid use and are assessed as likely to benefit from the reduction of opioid medication.

Consensus on reducing opioid dosage: There is consensus among U.S. health agencies regarding patients who wish to reduce, or stop, their opioid use. This consensus best practice approach to opioid reduction recommends a slow reduction of opioid medication. This approach of slowly reducing opioid use is called “tapering” and a series of slowly decreasing dosages is called a “taper.”

Long-standing need as relates to this patent application: The National Institute on Drug Abuse has indicated the need for a method of that will “allow an assessment of the treatment trajectory in patients under treatment for SUD [substance use disorder] and related conditions that are considered important to the mission of NIDA.” This invention will achieve the method sought by NIDA, by which it is possible to determine the treatment trajectory of patients with SUD. This application is for the invention of diagnostic opioid taper regimens. The regimens, each consisting of a sequence of dosages, can be administered to patients who are in various ranges of dosage dependencies. These regimens are designed to be used in the diagnosis of an optimal, or near optimal, course of treatment for a person who wishes to taper, i.e., reduce their opioid used. These regimens have a structure that is applicable to formulating diagnostic taper regimens for other medications that cause dependency, such as benzodiazepines.

Tapering is a focus of U.S. healthcare agencies: The U.S. Department of Health and Human Services (HHS), the Centers for Disease Control and Prevention (CDC), and the Veterans Health Administration (VHA) all discuss tapering in their publications. The 5-page “HHS Guide for Clinicians on the Appropriate Dosage Reduction or Discontinuation of Long-Term Opioid Analgesics” (2019) mentions tapers and tapering 70 times. The Guide states, “Tapers may be considered successful as long as the patient is making progress, however slowly, towards a goal of reaching a safer dose, or if the dose is reduced to the minimal dose needed.” The HHS Guide's “Opioid Tapering Flowchart,” has a step that advises, “Discuss, educate, offer taper, start slow taper when ready.”

A section in the HHS Guide is devoted to the importance of individualizing the rate of tapering, stating, “When opioid dosage is reduced, a taper slow enough to minimize opioid withdrawal symptoms and signs should be used. Tapering plans should be individualized based on patient goals and concerns. The longer the duration of previous opioid therapy, the longer the taper may take. Common tapers involve dose reduction of 5% to 20% every 4 weeks.” The HHS Guide does not present any method of determining a rate of tapering, be it optimal, clinically indicated or otherwise. This patent application, for Diagnostic Opioid Taper Regimens, addresses that gap by providing a method of determining an optimal, near optimal or otherwise clinically sound, rate of tapering.

In the 2022 CDC Clinical Practice Guideline for Prescribing Opioids for Pain, the terms “tapers” or “tapering” are mentioned 209 times. The Guideline states, that if continuing opioid therapy has more risks than benefits, then, clinicians should optimize other therapies and “work closely with patients to gradually taper to lower dosages or, if warranted based on the individual circumstances of the patient, appropriately taper and discontinue opioids.” No guidance is provided on the specifics tapering or how to determine a rate of tapering.

Veterans Health Administration Guidance: The VHA's “Pain Management Opioid Taper Decision Tool-A VA Clinician's Guide,” notes there are tapers reducing from “2 to 10% every 4 to 8 weeks with pauses in taper as needed,” . . . to “Consider for patients taking high doses of long-acting opioids for many years.” Options for reductions of “5 to 20% every 4 weeks with pauses in taper as needed” are also noted.

The VHA Opioid Taper Decision Tool notes, “Rapid tapers can cause withdrawal effects and patients should be treated with adjunctive medications to minimize these effects; may need to consider admitting the patient for inpatient care.”

The VHA Opioid Taper Decision Tool describes specific protocols in detail for tapering from a starting dosage of total daily dosage of 270 mg MEDD (morphine equivalent daily dose) extended-release morphine sulfate by a fixed amount of reduction of the total daily dose at one-month intervals. The 270 mg is taken in divided doses, as 90 mg three times/day. Options are described of tapering at various rates. Tapers employing monthly and weekly dosage reductions are described. The VHA Opioid Taper Decision Tool advises, “Continue the taper based on Veteran response. Pauses in the taper may allow the patient time to acquire new skills for management of pain and emotional distress while allowing for neurobiological equilibration . . . ” For the 2% to 10% taper, the Opioid Taper Decision Tool notes, “Continue following this rate of taper until off the morphine or the desired dose of opioid is reached.”

The VHA Opioid Taper Decision Tool has a section titled, “Considerations when formulating an opioid taper plan” and suggests, “Determine if the initial goal is a dose reduction or complete discontinuation.” The need to select the speed of the taper is noted, and rates of dosage reduction described in detail. However, there is no method suggested for determining the rate of tapering, even as many rates are mentioned. The Tool notes, “Slower, more gradual tapers are often the most tolerable and can be completed over several months to years based on the opioid dose. The longer the duration of previous opioid therapy, the longer the taper may take.” The VHA method of tapering that is presented is linear, with a fixed milligram amount of reduction per unit of time repeated until desired dosage level reached . . .

The recommendation of the Veterans Health Administration, in their subsequently published VA/DoD Clinical Practice Guideline (2022), as of evidence gathered through April 2021, is “There is insufficient evidence to recommend for or against any specific tapering strategies.”

HHS, CDC, and VHA emphasize the vital role of collaborative communication between patient and provider in all opioid-related treatments, including tapering. Behavioral counseling, along with social and medical support, are associated with improved tapering outcomes.

The CDC Guidelines, in a section on “Tapering Rate”, note, “Evidence to support specific tapering rates is limited.” The CDC Guidelines note the need to individualize “based on the patient's clinical situation.” Tapering should be slow enough to “minimize symptoms and signs of opioid withdrawal. Tapers can be completed over several months to years,” the Guidelines state that “Tapers of approximately 10% per month or slower are likely to be better tolerated than more rapid tapers when patients have been taking opioids for longer durations (e.g., ≥1 year).”

Abruptly reducing or discontinuing opioids, even at doses as low as 1 or 2 mg per day of buprenorphine, can result in severe withdrawal symptoms for most patients. HHS, CDC, and VHA guidance emphasize the importance of gradual tapering and avoiding sudden discontinuation.

Variability of Responses to Opioid Dosage Reduction: Patients who are dependent on opioids exhibit a considerable range of sensitivity to opioid dosage reduction. This variability to dosage reduction is a major consideration of the VHA “Pain Management Opioid Taper Decision Tool” which references a broad range of tapering rates. The Decision Tool categorizes patients in terms of the rates at which various patients are capable of reducing their opioid usage. Some of the ranges that are discussed range from 2% per eight weeks to 20% per month.

Tapering has risks. Risk/Benefit Analysis Required: Tapering opioids carries risks that are greater than the risks for patients who are maintaining their opioid dosage at a consistent level in an opioid maintenance program. Patients who taper must first be evaluated to determine if a taper is justified and if the benefits outweigh the risks. In a study of 199,836 individuals “opioid tapering was associated with a small absolute increase in opioid overdose or suicide compared with a stable opioid dosage” (Larochelle et al. 2022).”

Practical Problem: One significant challenge faced by patients and clinicians involved in tapering is the lack of commercially available doses that would enable tapering. There are no dosage sequences designed or available that would enable a tapering process aligned with HHS, CDC and VHA guidelines. The most widely prescribed buprenorphine medication for opioid maintenance programs in the United States is Suboxone® (Indivior), with doses of 2, 4, 8, and 12 mg formulated as films. Orexo manufactures Zubsolv® buprenorphine tablets with bioavailability of 1, 2, 4, 8 and 12 mg (Orexo states amounts of buprenorphine HCL are 0.7; 1.4; 2.9; 5.7, and; 8.6 mg and equate to the bioavailable amounts). These products contain naloxone in 1:4 ratio to buprenorphine to deter diversion. Tablets without naloxone are available. The tablets are more difficult to divide precisely than the films.

The intervals between the commercially available dosages are too great to permit effective tapering for most patients. For example, a 12 mg to 8 mg decrease requires a 33% decrease. From 8 mg to 4 mg or from 4 mg to 2 mg would each represent a 50% decrease. Dosage reductions of this magnitude greatly exceed consensus guidelines for tapering and are at rates associated with emergence of severe withdrawal symptoms.

Dividing existing formulations: At dosages for which there are commercial products consisting of an 8 mg sublingual film (e.g., Suboxone®) or 8 mg sublingual tablet (e.g., Subutex®), some patients attempt to make their own dosage reduction tapers by fashioning a series of decreasing dosages of their medication, although this is against the prescribing guidelines published by manufacturer. At dosages for example, of 8 mg and 4 mg, some patients, those not very sensitive to dosage decreases, may be able to divide their doses in such a manner as to successfully taper. The more sensitive to dosage reduction that a patient is, the more difficult this approach to a patient making their own dosages would be. If dividing a tablet or film produces a dose too small, the patient would be at greater risk of a withdrawal reaction. A few patients can self-taper with a 2 mg dose. These efforts lack precision and are not suitable for many, perhaps most, patients. Patients dividing doses and taking their medication and taking divided doses throughout the day is relatively common for patients who are in drug maintenance programs. In those situations, because of the relatively long half-life of a medication such as buprenorphine, if the patient consumes the prescribed amount in a 24-hour period, the fact that the doses were unevenly divided will have only a marginal effect. When a taper of 10% vs a taper of 5% differs by less than a tenth of a milligram, it is unrealistic to expect a patient to achieve these levels of precision. Since every taper eventually passes through the range of 2 mg to zero, the fact that there are not commercial tapers becomes significant in almost every case.

In summary, the commercial dosages of 1, 2, 4, 8 and 12 mg do not offer a realistic path for precision tapering for most patients. Some patients may be able to taper from 12, 8, and 4 mg dosages down to 2 mg. However, the approximations that will occur may result in the more sensitive patients being unable to tolerate any dosage decreases that happen to be too great.

Some specifics of the challenge of patients dividing dosages so as to taper: In the United States, the 2 mg dose of buprenorphine is the lowest dosage made by the major manufacturer, Indivior. The 0.7 mg dose made by Orexo, has a bioavailability of 1 (one) mg of buprenorphine. For a patient who can divide these dosages into halves and quarters some options for tapering may be possible. However, if arithmetic adherence to a taper down to a range such as 0.5 mg and below is necessary, for example, with a patient relatively sensitive to dosage reduction, then the precision required will not be possible without a laboratory balance and some laboratory technical skills.

What the patient confronts when going to divide doses: The 2 mg Indivior film (Suboxone®) is ⅞ by ½ inch (22.0×12.8 mm). Some patients can take this 2 mg film and make strips containing 1 mg of buprenorphine, which would be approximately ½ by ½ inch (actual: 7/16″×½″). To create a 0.5 mg dose, it would require making a piece of film approximately ¼ by ½ inch (actual ¼″× 7/16″). For many patients this is not a practical option and the precision required would be difficult, or not possible, for most patients. Significantly, patients and providers would not know with requisite accuracy the amount of a dose that was consumed.

Extended-release injectable buprenorphine: Sublocade® and Brixadi® are formulations of extended-release injectable buprenorphine that are used for opioid maintenance programs. Upon injection, a small crystalline depot forms. As the depot dissolves over time, buprenorphine is released. A peer-reviewed journal article reports on a single Sublocade® injection administered to each of three patients who had been consuming prescribed doses of 2 to 6 mg/day Suboxone® for some years. Following the single injection of 100 mg of Sublocade® the patients no longer required further buprenorphine in any format nor did they report cravings. (Ritvo et al. 2021). A large study currently enrolling (NCT04464980) aims, in part, to determine efficacy of tapering with extended-release injectable compared with tapering with sublingual films.

Importance of Low Dosage tapering: One major reason low dosages acquire great importance in tapering is because, as noted above, it is not possible for patients who are seeking to stop their opioid usage to do so without passing through these low dosage ranges prior to reaching zero mg of buprenorphine. It is well-established among patients, and addiction medicine practitioners, that the last few milligrams of an opioid taper, e.g., from approximately 2 mg to zero mg of buprenorphine reduction, is often the most difficult for a patient who is trying to taper to abstinence.

Applying accepted tapering theory to dosage reduction: As mentioned above, CDC notes that “Evidence to support specific tapering rates is limited.” There is currently no research supporting the notion that reducing the dosage of a medication from a certain level to zero requires specific gradual reductions over time or that one set of dosage reductions is superior to another. In a linear (each decrease same milligram amount) taper from 2 mg in which each dosage step down, each dosage reduction, (decrement) is 0.2 mg, the required sequence of dosages from 2 mg to 0 (zero) mg is 2.0; 1.8; 1.6; 1.4; 1.2; 1.0; 0.8; 0.6; 0.4; 0.2 mg, to reach 0 mg.

In a taper in which each Step reduced by 10% of the continually changing most recent Step (an exponential taper), starting at 2 mg, would require 28 steps to reduce from 2.0 to 0.10 (one-tenth) of a mg. The first five and last five steps would be 1.80; 1.62; 1.46; 1.31; 1.18 . . . (18 steps) . . . 0.16; 0.14; 0.13; 0.12, and; 0.1 mg.

Commercial Dosages for Tapering are not manufactured: With either the linear or the exponential taper, it becomes clear that dosages do not currently exist for patients to taper. At these dosages, the difference between a tolerable decrease and an intolerable decrease would at some points be measured in hundredths of a milligram. For example, a 5% taper from 1.0 mg results in the next dose being 0.95 mg. A 10% taper from 1.0 mg results in the next dose being 0.90 mg. The difference between 0.95 and 0.90 is 0.05 mg, i.e., 1/20 of a milligram. It can be difficult to accept that the distinctions between a 5% and 10% taper from 1 mg are experienced by patients who are tapering. It is easier to accept that some patients can taper from 10 mg to 9.5 mg but might have a problem tapering from 10 mg to 9 mg all at one time. Decreasing by 1 mg, all at one decrease, highlights the reality that all tapers can be radically slowed by dividing any decrement into smaller decrements. For example, a patient might be able to taper from 10 mg to 9 mg in four weeks if the steps down were 0.25 mg weekly and not 1.00 mg all at Day 29.

Accepting that tapering could, in fact, be accomplished at these low dosages, and that these distinctions of various tapering rates are valid, would require a re-thinking what is currently produced commercially, if there was an objective of producing tapers. One school of thought is that effective linear tapers can be achieved at lower dosages and that “percentage of a percentage” (exponential) tapers, are not required at lower dosages.

Formulation of tapers vs. formulation of diagnostic tapers: This patent application is for diagnostic opioid tapers. A previous patent issued this applicant (JLG), U.S. Pat. No. 11,253,512 B2, is for a method to make opioid tapers. The method of making the Diagnostic Opioid Taper Regimens (DOTRs) is addressed in this application. U.S. Pat. No. 11,253,512 B2 describes another way to make any tapers described herein and any dosage sequence in which a high degree of precision dosage formulation is required.

Advantages gained by usage of DOTRs: Determining the optimal rate of tapering can shorten treatment time for a patient. Shorter treatment time confers significant utility on the invention. Changes in the rate of tapering will always result in changes in the time to complete a taper if all other variables held constant. To reduce from 2 (two) mg to 0.2 mg at a 10% exponential rate requires 22 steps down (decrements). Reducing from 2 mg to 0.2 mg at a 5% exponential taper requires 45 steps down (decrements). This is with all steps being of the same duration.

A linear taper of 10% of with a starting 2 mg dose, i.e., with a series of 0.2 mg decreases (decrements), requires 20 steps. A linear taper of 5% of the original 2 mg dose, i.e., utilizing a 0.1 mg decrement, requires 40 steps. Therefore, determining the highest rate of tapering at which a patient is still comfortable, i.e., no significant withdrawal symptoms is meaningful. “Significant” withdrawal symptoms here means a withdrawal symptom strong enough to make the patient want a higher dose of opioid. Not addressed in these examples is the final 0.2 mg of tapering, from 0.2 to zero mg. For some patients this final 0.2 mg may offer no challenge and some patients can simply stop. For other patients they may find that some level of opioid medication is required for a period of time exceeding that predicted by the initial exponential or linear plot of dosage vs. time. In other words, an adjustment in the rate of dosage reduction may be required, even if many reductions at a specific rate have already been accomplished.

NIDA seeks diagnostic tool: As noted above, this application for Diagnostic Opioid Taper Regimens is for an invention designed to be able to be what NIDA references in a 2022 omnibus grant solicitation, that is something that will “allow an assessment of the treatment trajectory in patients under treatment for SUD [substance use disorder] and related conditions that are considered important to the mission of NIDA.”

There are no diagnostic tapers: There are no commercially produced diagnostic tapers. There are no commercially produced dosages, or sequences of dosages, that are designed to determine, or lend themselves to determining, a treatment trajectory for a patient with a substance use disorder. In order to determine a patient's optimal taper rate, it is necessary to first create the rate of dosage reduction optimal for that patient.

Those clinicians who seek to determine the optimal rate of tapering currently must use a trial-and-error method in which patients divide their commercial doses. Except for the circumstance of patient and clinician deciding to utilize a compounding pharmacy, the attainment of a high level of precision in trying to determine an optimal rate of tapering would likely prove elusive.

If efficacy should be demonstrated: Should this application's, “Diagnostic Opioid Taper Regimens” prove efficacious in randomized controlled trials, these Diagnostic Opioid Taper Regimens would make diagnosing the trajectory of a patient a far more orderly and time efficient process than currently. This invention would fill the gap in the field of addiction medicine represented by a lack of capacity to effectively diagnose opioid tapering rates. This gap is now encountered by clinicians who attempt to determine an optimal, or near optimal, rate of tapering. In theory, if optimal tapering rates were routinely determined, then treatment times would be significantly shortened for those patients for whom tapering is both physiologically possible and beneficial.

DOTR is novel, nonobvious and answers long-standing need: Each of the individual diagnostic opioid taper regimens, with their varied starting points and varied rates of dosage reduction, are novel both in concept and in their embodied physical form. This is particularly true as regards the aspect of this diagnostic regimen involving accelerating the rate of dosage decrements from one decrement step to the next decrement step. That is, particularly as the rate of tapering grows more rapid with the passage of time.

Part of the novelty arises from the relationship of the proportions of sequential dosages within a taper. Taken as a whole, the diagnostic opioid tapers are novel, considered either conceptually or as an embodiment. The idea of the accelerating rate of tapering does not appear likely to have occurred as an idea to persons of ordinary skill in the art. The idea of embodying a sequence of dosages of increasing rates of tapering is inventive. This is especially so since most practitioners likely still consider it unrealistic to make the dosages required and would therefore be even more unlikely to consider a solution that requires dosages that do not exist. Even if the definition of one of ordinary skill in the art were expanded, this idea seems unlikely to have occurred and appears not to be mentioned in any materials it has been possible to find. One method of making dosages with small decrements between the dosages was patented in U.S. Pat. No. 11,253,512 B2 and another method of manufacture is described herein. That said, without being aware that it is practical to make the small dosages and to achieve small decrements it is not clear how a person having ordinary skill in the art would begin to conceptualize the nature of the details of a diagnostic opioid taper regimen. The acceleration of the rate of taper of the decreasing dosages is an additional step to the practical necessity of making the micro-decrements that would need to occur in rather some detail. Then there is the issue of even if a person skilled in the art had the general idea would they have the arithmetic skills to express the concept in the detail required? Would they have the capacity to write the section “Method of Making the Invention”? Given the reality that this concept in nowhere discussed, and the term of “diagnostic opioid taper” appears nowhere on the Internet, are arguments for nonobviousness.

This Diagnostic Opioid Taper Regimen answers a long-sought need: The DOTR provides a way to determine the best rate of tapering for patients who wish to taper. Or, as per a NIDA research priority, “allow an assessment of the treatment trajectory in patients under treatment for SUD [substance use disorder] and related conditions that are considered important to the mission of NIDA.”

Utility of invention as a research tool: The nonobviousness of the invention becomes more pronounced when considering its qualities as a research tool. When the unique qualities, particularly of the exponential tapers that are part of the various regimens are considered, it becomes clear that this tool will be very helpful in answering long-standing questions about the comparative effectiveness of various tapers.

Background in context of history of opioid dependency treatment in US: Medical treatment of opioid addiction and dependency has a relatively long history in the United States. In the 1930's, there was a shift from a criminalized view of opioid dependency towards treating opioid dependency, then referred to almost exclusively as opioid addiction, as a medical condition requiring treatment.

Treatment with the following medications has focused on maintenance in which the goal is to prevent drug cravings and prevent a return to unprescribed drugs. In the 1960's the medical use of methadone was introduced when it was established that methadone was capable of freeing patients from opioid cravings. In 2000, the U.S. Congress approved the use of buprenorphine to treat opioid dependency and addiction by practitioners with a special license. In 2021, general practitioners without the special license were approved to treat 30 patients or less.

The formulation of buprenorphine through 2018 was in sublingual and buccal doses. In 2018, an extended-release injectable formulation, Sublocade®, was introduced for monthly injections. In 2023, Brixadi® was approved. Brixadi® has weekly and monthly formulations.

Randomized controlled trials have not determined effective methods of tapering opioids. Historically, the number of patients who have been shown to succeed in tapering to opioid abstinence in a given trial is approximately 10%, or slightly more. A 2022 Lancet-Stanford study found approximately 10% per year of patients in opioid maintenance discontinue their opioid medication (Rao). There is research interest in extended-release injectable buprenorphine for its potential to be effective in the tapering process. Approximately half of all patients in opioid maintenance programs return to use of unprescribed opioids.

The patent previously issued this applicant, JLG, U.S. Pat. No. 11,253,512 B2, describes how to make tapers that align with the guidelines recommended by the U.S. Department of Health and Human Services and the Centers for Disease Control and Prevention.

The invention addressed in this instant application, Diagnostic Opioid Taper Regimens, describes how to make a medication sequence will “allow an assessment of the treatment trajectory in patients under treatment for SUD [substance use disorder] and related conditions . . . ” As noted above, there are no opioid dosage sequences designed or manufactured for tapering (other than 11,253,512 B2) nor for determining the rate of a clinically sound taper.

If the invention in this application is proven effective in randomized controlled trials, the invention will constitute a significant advance in the diagnostic tools available to the field of addiction medicine. The mechanisms, structure, and method of making this invention will also be applicable to addressing issues of diagnosing the treatment trajectory of patients who elect to discontinue other drugs capable of causing dependency and addiction. It seems probable that diagnostic tapers formulated with benzodiazepines or selective serotonin reuptake inhibitors (SSRIs) may offer a potential solution for diagnosing and determining the treatment path for patients who choose to taper off these medications.

### PRIOR ART

This invention is a diagnostic opioid taper regimen. There are no prior diagnostic opioid taper regimens, either in name or in fact. Health care providers try to determine the optimal course of treatment for a patient who is reducing their opioid dosage. However, there is no fixed protocol that attempts to determine a clinically sound course of dosage reduction. The National Institutes on Drug Abuse stated in 2022 they would welcome some protocol that was able to show the trajectory of treatment of a patient with drug dependency. Neither of the terms “diagnostic opioid taper regimens” or “diagnostic opioid taper” are found in patent searches nor in an internet search, as of Sep. 16, 2023. Key ideas that comprise this invention are nowhere found to have been proposed previously, such as accelerating rate of dosage reduction and reductions at different specific rates.

The prior art that exists has to do with tapering. However, none of the art concerns determining the best way to taper other than knowing something about the patient's current dosage level and history. There is no prior mention of a diagnostic taper to determine a rate of tapering nor an optimal rate of opioid tapering

Tapering studies include, but are not limited to, Neilson et al. (2013) “A comparison of buprenorphine taper outcomes between prescription opioids and heroin users.”; Woody et al. (2008), Extended versus short-term buprenorphine-naloxone for treatment of opioid addicted use—a randomized trial “; Sigmun et al. (2013) “Randomized, Double-blind Evaluation of Buprenorphine Taper Duration in Primary Prescription Opioid Abusers.” These studies are instructive in that they show the problems encountered when there is no protocol to determine treatment courses among patients and the high failure rates that occur when trying a uniform approach with all subjects.

Patents cited previously in this applicant Grossman's U.S. Pat. No. 11,253,512B2 demonstrate the issues that can arise when there is no attempt to determine a treatment trajectory. Examples are, Reidenberg et al., U.S. Pat. No. 8,637,073; Peroutka, patent Ser. No. 12/952,823, and Pergolizzi, US2012/037619. None of these suggest a diagnostic approach that could be considered to resemble instant application.

It is not apparent that any of the previous work would teach, suggest or motivate a person skilled in the art to make the inventive steps of first, realizing it is possible to make the small dosage increments that are essential and that align with the tapering guidelines of U.S. Department of Health and Human Services and Centers for Disease Control and Prevention. The protocol of exposing a substance-dependent person to a sequence of tapers that are increasing in velocity is nowhere found previously While in hindsight, some might say it is obvious, the degree to which the subject of exposing a patient to a series of increasingly rapid tapers is nowhere mentioned or suggested in the field of addiction medicine, to the best of this applicant's knowledge. This would appear to support a conclusion of nonobviousness. In addition, once the concept of accelerating the tapers comes to mind, it leads to the exploration of the mechanics and mathematics required to develop what is essentially a mini-formulary for diagnosing varying degrees of opioid dependency. The math and the mechanics involve figuring out how to structure the regimen and then applying this idea to various regimens. The evidence for this can be found in each of the graphs and tables presented in Drawings.

The absence of the invention in prior art coupled with a “long-felt but unsolved need” for a way to determine the treatment trajectory appears to be objective indica that supports nonobviousness. The failure of others to develop a similar solution is further objective indica supporting nonobviousness.

The potential for commercial success is another factor to consider. There are a large number of potential patients. There are 2.3 million individuals with opioid dependency in the U.S. with about 670,000 receiving medications for opioid use disorder and opioid dependency. Some significant number of these patients will seek to reduce or stop their medication. Furthermore, the diagnostic taper regimen concept is likely to be readily adapted to medications such as benzodiazepines and anti-depressants. While the profiles of trying to stop the use of benzodiazepines and anti-depressants are different, there would appear to be significant similarities. Estimates show larger numbers of people dependent upon benzodiazepines and anti-depressants than on opioids. “Treatment trajectory” may be seen as a synonym for “a clinically sound course of treatment” and, as NIDA indicates, there are currently no methods for distinguishing among substance use disorder patients in such a way as to consistently chart differential treatment trajectories.

### REFERENCES

Dowell D, Jones C, Compton, W et al. HHS Guide for Clinicians on the Appropriate Dosage Reduction or Discontinuation of Long-Term Opioid Analgesics US Dept HHS 2019
Dowell D, Ragan K R, Jones C M, Baldwin G T, Chou R. CDC Clinical Practice Guideline for Prescribing Opioids for Pain—United States, 2022. MMWR Recomm Rep 2022; 71 (No. RR-3): 1-95.
Greenwald, M. K., Comer, S. D., & Fiellin, D. A. (2014). Buprenorphine maintenance and mu-opioid receptor availability in the treatment of opioid use disorder: implications for clinical use and policy. Drug and alcohol dependence, 144, 1-11. https://doi.org/10.1016/j.drugalcdep.2014.07.035
Himstreet J, Popish S, Robeck I, Saenger M. Pain Management Opioid Taper Decision Tool, A VA Clinician's Guide, US Department of Veterans affairs, Veterans Health Administration, PBM Academic Detailing Service
Larochelle M R, Lodi S, Yan S, Clothier B A, Goldsmith E S, Bohnert A S B. Comparative Effectiveness of Opioid Tapering or Abrupt Discontinuation vs No Dosage Change for Opioid Overdose or Suicide for Patients Receiving Stable Long-term Opioid Therapy. JAMA Network Open. 2022; 5 (8): e2226523. doi: 10.1001/jamanetworkopen.2022.26523
National Institute on Drug Abuse. SBIR/STTR Topics of Interest. Biomarker Development for Substance Use Disorders (SUDs). See: Biomarker Development for Substance Use Disorders (SUDs) https://nida.nih.gov/funding/small-business-innovation-research-sbir-technology-transfer-sttr-programs/about-sbir-sttr-programs/topics-of-interest Accessed Aug. 18, 2023.]
National Institute of Environmental Health Sciences, National Institutes of Health. Health and Education, Biomarkers. https://www.niehs.nih.gov/health/topics/science/biomarkers/index.cfm
Rao I J, Humphreys K, Brandeau M L. Effectiveness of Policies for Addressing the US Opioid Epidemic: A Model-Based Analysis from the Stanford-Lancet Commission on the North American Opioid Crisis. The Lancet 2021
Ritvo A D, Calcaterra S L, Ritvo J I. Using Extended Release Buprenorphine Injection to Discontinue Sublingual Buprenorphine: A Case Series. J Addict Med. 2021 May-June 1; 15 (3): 252-254. doi: 10.1097/ADM.0000000000000738. PMID: 32925232.
U.S. Department of Health and Human Services PHS 2022, Omnibus Solicitation of The National Institutes of Health, Centers for Disease Control and Prevention, and Food and Drug Administration for Small Business Innovation Research (SBIR) and Small Business Technology Transfer (STTR) Grant Applications; NIH, CDC, and FDA Program Descriptions and Research Topics p 61
VA/DoD Clinical Practice Guideline. (2022). Use of Opioids in the Management of Chronic Pain Work Group. Washington, DC: U.S. Government Printing Office. Pgs. 34, 52, 127.

### BRIEF SUMMARY OF THE INVENTION

The invention comprises a sequence of slowly decreasing dosages of buprenorphine. The dosages decrease in a specific manner in which the rate of decrease is precisely controlled. The invention is a method of determining a clinically sound course of treatment for patients in medication maintenance programs who wish to reduce, or stop, opioid use.

The rate of dosage reduction grows larger each time the dosage is reduced. This increasing rate of dosage reduction—i.e., accelerating rate of dosage reduction—allows the patient to experience a sequence of increasingly rapid dosage reductions. For example, reducing the dose by 4% at one decrement and by 5% at the next decrement.

Patients show a wide variability of reactions to the reduction of opioid medication. When the rate of dosage reduction exceeds the rate at which a patient is comfortable reducing their dosage, the patient will start to experience symptoms of withdrawal.

The emergence of symptoms of withdrawal is indicative of the maximum rate of dosage reduction at which the patient is comfortable. The emergence of withdrawal is intended to be a signal that enables the provider to prescribe timely a taper of an appropriate rate. The starting dosage and the rate of dosage reduction of the diagnostic opioid taper can be varied to align with a patient's history and tapering requirements. The diagnostic taper is designed to be utilized with any medication that can cause dependency, such as benzodiazepines or selective serotonin reuptake inhibitors (SSRIs).

The advantage this invention offers is it will enable a significant number of patients currently in opioid treatment, who desire to reduce or stop their opioid use, to successfully achieve this objective. It is probable it will enable more patients to stop or reduce their opioid use than currently is possible. To be considered an appropriate candidate for opioid reduction a patient must be assessed as likely to benefit from opioid dosage reduction. This invention solves previously existing problems by creating a needed diagnostic regimen of medication where no such regimen currently exists.

Practitioners often treat patients who decide to reduce or stop their opioid medication and can benefit from such reduction or cessation. One of the first steps is to determine the best course of dosage reduction for such patients. A provider treating a patient who is qualified to taper, and who elects to taper their opioid medication, tries to diagnose the best rate at which their patient can reduce their medication. This invention comprises the very specific sequence of dosages, and timing of dosage reduction, that is required to diagnose a clinically sound course of treatment by which that patient can reduce or stop their opioid medication.

The invention requires the patient and provider to work cooperatively and to communicate effectively. The patient must notify the provider when certain specified symptoms of opioid withdrawal emerge.

When that symptom of opioid withdrawal, that the patient and clinician have discussed, is experienced by the patient, it is taken as a signal that the Diagnostic Opioid Taper Regimen has reached a velocity of opioid reduction that is a bit too fast to be sustained by that patient. At that point, or by prearrangement, patient and provider decide upon a course of treatment that both believe will be effective. One likely candidate rate for the patient's taper at this point would be the immediately prior rate of tapering in the Diagnostic Opioid Taper Regimen during which the patient did not have their “signal” symptom.

SUMMARY: The Diagnostic Opioid Taper Regimen is a diagnostic tool intended to be used by prescription with patients who are suitable candidates for reducing or stopping opioid medication. Proper use requires a high level of effective communication between patient and provider. At some point during administration of the taper, symptoms of opioid withdrawal start to emerge. This is a signal to the patient and the provider that it is likely that the optimal rate for tapering has just been exceeded. The patient and provider then agree upon a rate of opioid tapering that appears suitable to patient and provider.

### BRIEF DESCRIPTION OF THE DRAWINGS

FIG. 1A: “Diagnostic Opioid Taper Regimen, Baseline 1 mg, 1% to 10% Step decreases,” shows graph of a Diagnostic Opioid Taper Regimen with a Baseline (i.e., starting dosage) of one milligram and followed by ten successive Steps. On the y-axis of graph is buprenorphine, in milligrams, and on the x-axis are Baseline and numbered Steps, with Baseline and Steps being periods of time.

FIG. 1B: Both FIGS. 1A graph and 1B table show dosage level for Baseline and each Step and the percentage amount (1% through 10%) by which each Step dosage is less than the previous Step dosage.

FIG. 2A: This is graph of “Diagnostic Opioid Taper Regimens, Baselines of 0.5, 1.0, 2.0, 4.0 and 8.0 milligram, 1% to 10% Step decreases”. Graph in FIG. 2A shows Diagnostic Opioid Taper Regimens for patients starting at 0.5, 1.0, 2.0, 4.0 and 8.0 milligrams. The rate of tapering for all Regimens, across all Baselines (starting dosages), increases by 1% at each of 10 Steps.

FIG. 2B: Same information in tabular form as FIG. 2A shows as graph.

FIG. 3A: Shows in graphic form “Diagnostic Opioid Taper Regimens, Baselines of 0.5, 1.0, 2.0, 4.0 and 8.0 milligrams, 2%, 4%, 6%, 8% and 10% Step decreases.” Shows Diagnostic Opioid Taper Regimen for patients starting at 0.5, 1.0, 2.0, 4.0 and 8.0 mg. Rate of tapering increases at Steps 1, 2, 3, 4, 5, by 2%, 4%, 6%, 8% and 10% respectively and thereafter continues at 10% dosage reduction each Step thereafter.

FIG. 3B: Same information in tabular form as FIG. 3A shows as graph.

FIG. 4. Table 4A shows the amounts of buprenorphine and excipients to make Diagnostic Opioid Taper Regimens for a DOTR that administers a 1 milligram tablet. Table 4B shows the amounts of buprenorphine and excipients to make Diagnostic Opioid Taper Regimens for a DOTR that administers tablets of 250 mg four times per day. Both Table 4A and 4B show for DOTR dosage decreasing at each Step by 1%.

### DETAILED DESCRIPTION OF THE DRAWINGS

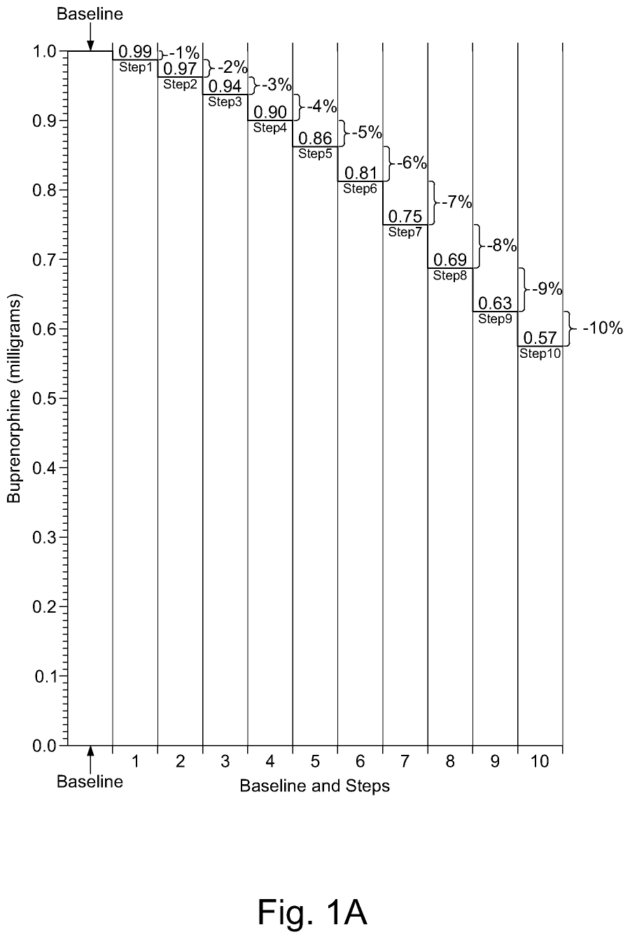

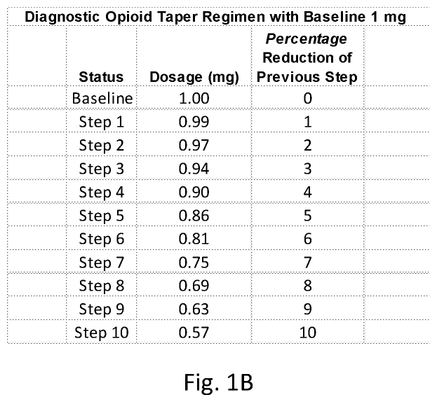

FIG. 1A and FIG. 1B: FIG. 1A and FIG. 1B show the same data in a graph and a table, respectively. FIG. 1A graph title is “Diagnostic Opioid Taper Regimen, Baseline 1 mg, 1% to 10% Step decreases.” FIG. 1A and FIG. 1B show the dosages administered in a Diagnostic Opioid Taper Regimen with a Baseline (starting dosage) of 1 milligram and 10 (ten) successive Steps. On the y-axis of FIG. 1A is buprenorphine, in milligrams, and on the x-axis are Baseline (starting dosage) and Step numbers. In the embodiments discussed in the Specifications, the duration of Baseline and the duration of each Step is four weeks. FIG. 1A and FIG. 1B show dosage level for Baseline and each Step and the percentage amount by which each Step dosage is less than the previous Step dosage in graph and tabular format. The time duration of the Steps is not shown in FIG. 1A and FIG. 1B. Duration can be at discretion of provider or investigator.

FIG. 1B table shows the amount by which each Step dosage is less than the previous Step in column headed as “Percentage reduction of previous Step.” FIG. 1A and FIG. 1B also show the buprenorphine dosage of each Step, in milligrams. In FIG. 1A the dosage amount is shown on (the line representing) each Step, e.g., on Step 1 is written, “0.99”. In FIG. 1B, the table, the dosage for each Step is given in column with heading “Dosage (mg)”.

The percentage by which each Step's dosage is reduced, compared to previous Step, is shown in FIG. 1A and FIG. 1B. In FIG. 1A graph, to the right of vertical component at the end of each Step there is a number followed by a “%” sign. The vertical component connects two successive Steps, for example, Steps 3 and 4. This number, in the case of Step 3 to Step 4 being “−4%” represents the percentage by which the successive (the immediately following) Step 4 is reduced compared to the immediately previous Step 3. As another example, in FIG. 1A graph, if locate Step 1 on x-axis and look at dosage level, will see 0.99 mg as the dosage on the Step (the horizontal component). Next to the vertical component there is a number “−1” followed by a % sign. This is the amount, “−1%”, that Step 1 has reduced compared to the immediately previous Baseline. Another example: If locate Step 10 will see dosage amount on Step 10 on Graph 1 is 0.57 mg and an amount of decrease from Step 9 to Step 10 of “−10%.” The same data in FIG. 1A graph is presented in FIG. 1B, the table.

In addition to showing a diagnostic opioid taper regimen with a Baseline of 1 mg, and acceleration of 1%, the graph of FIG. 1A shows the overall shape of a Diagnostic Opioid Taper Regimen (DOTR) in general. All DOTRs have a series of Steps, each one of which is followed by a percentage decrease in the dosage amount. The dosage remains constant for the duration of each Step.

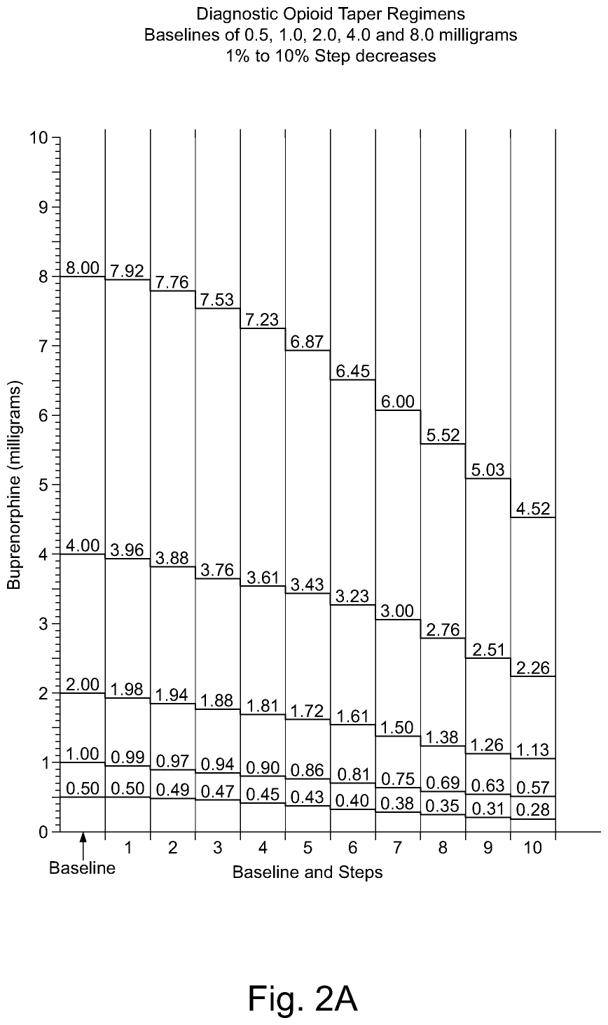

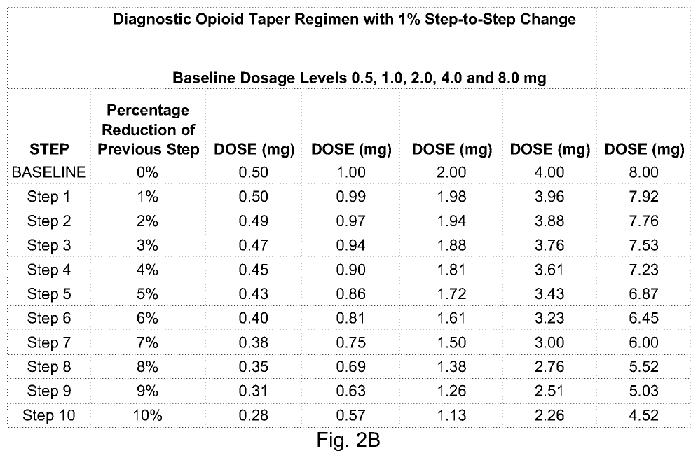

FIG. 2A and FIG. 2B: The graph and table of FIG. 2A and FIG. 2B show the same data: “Diagnostic Opioid Taper Regimens, Baselines of 0.5, 1.0, 2.0, 4.0 and 8.0 milligram, 1% to 10% Step decreases”. FIG. 2A and FIG. 2B show Diagnostic Opioid Taper Regimens for patients starting at 0.5, 1.0, 2.0, 4.0 and 8.0 milligrams. The rate of tapering for all Regimens in FIG. 2A and FIG. 2B increases by 1% at each of 10 Steps. On the y-axis of FIG. 2A graph is buprenorphine, in milligrams, and on the x-axis are Baseline and Step numbers.

These DOTRs may be used starting at any of these (0.5, 1.0, 2.0, 4.0 and 8.0 mg) dosages, dependent upon the dosage level at which a patient has stabilized in their opioid maintenance program. The Regimens shown in FIG. 2A and FIG. 2B are all proportional. This means that the reduction from a specific Step to the next Step, for example from Step 1 to Step 2 in every Regimen, is the same percentage decrease as that same Step number to the next Step number in every other DOTR in FIG. 2A and FIG. 2B. Research may show that changing the parameter of percentage change from one Step to the next Step may have an advantage diagnostically.

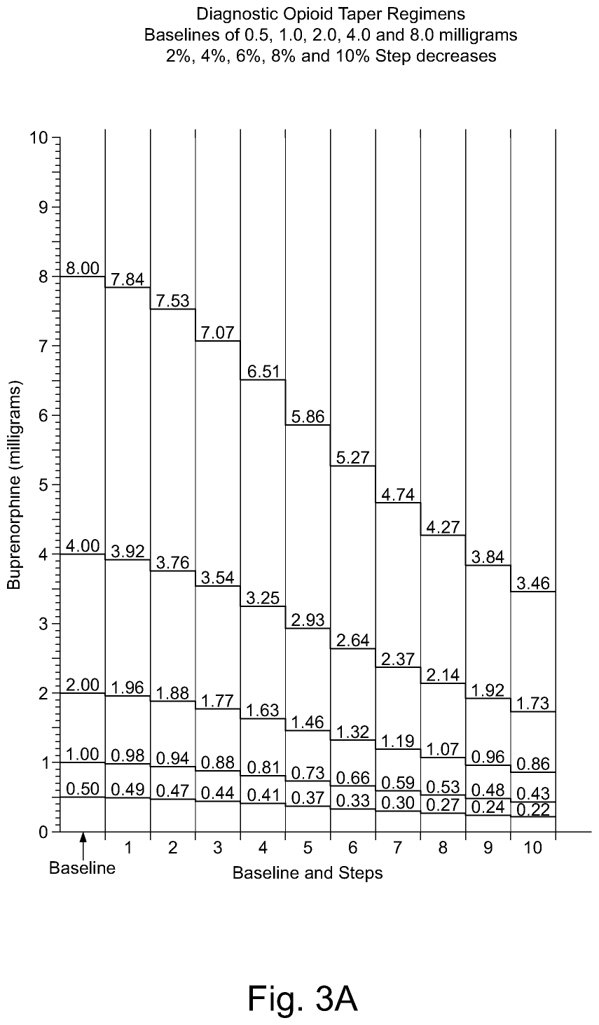

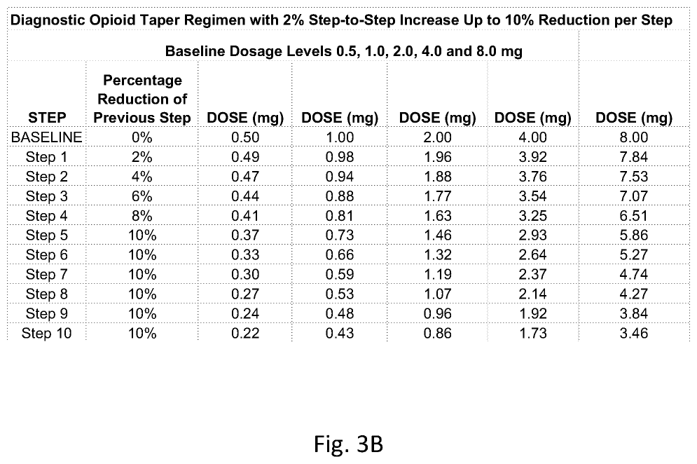

FIG. 3A and FIG. 3B: “Diagnostic Opioid Taper Regimens, Baselines of 0.5, 1.0, 2.0, 4.0 and 8.0 milligrams, 2%, 4%, 6%, 8% and 10% Step decreases” shows Diagnostic Opioid Taper Regimens for patients starting a diagnostic taper at 0.5, 1.0, 2.0, 4.0 or 8.0 mg and tapering at a rate in which the dosages are decreasing by an additional 2% at each of first five Steps. This results in tapers of 2%, 4%, 6%, 8% and 10%, respectively, each lasting a period determined by investigator or practitioner, but here intended as 4 weeks. This is an example of how the parameter of the rate of dosage reduction can be varied. Compare with the 1% increase per Step in FIGS. 1A, 1B, 2A and 2B.

After the first five Steps, each Step then decreases by 10%. By the 10th Step, the dosage has reduced by 57%, compared to Baseline. If the Steps are each of 4 weeks duration, the 10th Step starts at the 37th week. The DOTR is designed to identify optimal taper rates for most patients significantly prior to the 10th Step.

FIGS. 3A and 3B demonstrate the broad range of starting dosages within which the Diagnostic Opioid Taper Regimens can operate. Here we see the DOTR being useful for patients coming out of maintenance with a range of total daily dosages. The rates of dosage decrease shown in FIGS. 3A and 3B would be more likely to be utilized with patients who had had relatively brief exposure to opioids but had become opioid-dependent, e.g., following an opioid prescription for two months following hip surgery. In such a circumstance, the practitioner would have a high index of suspicion that the patient was not as dependent as a patient with years of, for example, a relatively high dosage of opioid for 10 years. “As dependent” here means that the patient with shorter or less exposure might not need years of tapering with buprenorphine, and in fact might need a period of weeks to months. VHA Opioid Taper Decision Tool references this phenomenon.

For example, shown in FIGS. 3A and 3B, after four Steps, each of four weeks duration, i.e., at 16 weeks, at the start of the 5th Step, the patient dosage is at a dosage that is 27 (twenty-seven) % less than at the start of their Baseline dosage. The most recent prior tapering rate from Step 4 to Step 5 has a velocity of 10% with the patient reducing from 0.81 mg to 0.73 mg.

Some practitioners remain skeptical that these minor reductions could represent the highest pace at which a patient can tolerate a reduction in opioid dosage. Given the considerable variability in individuals' sensitivity to opioid medication reduction, it is likely that this variability would be present to some significant degree among a large group of patients. That is, some patients would easily reduce from 0.81 mg to 0.73 mg while some would not. There is also a reasoned school of thought that at this low dosage level there are finite milligram reduction amounts that can be repeated at the same milligram amount to produce a successful linear taper.

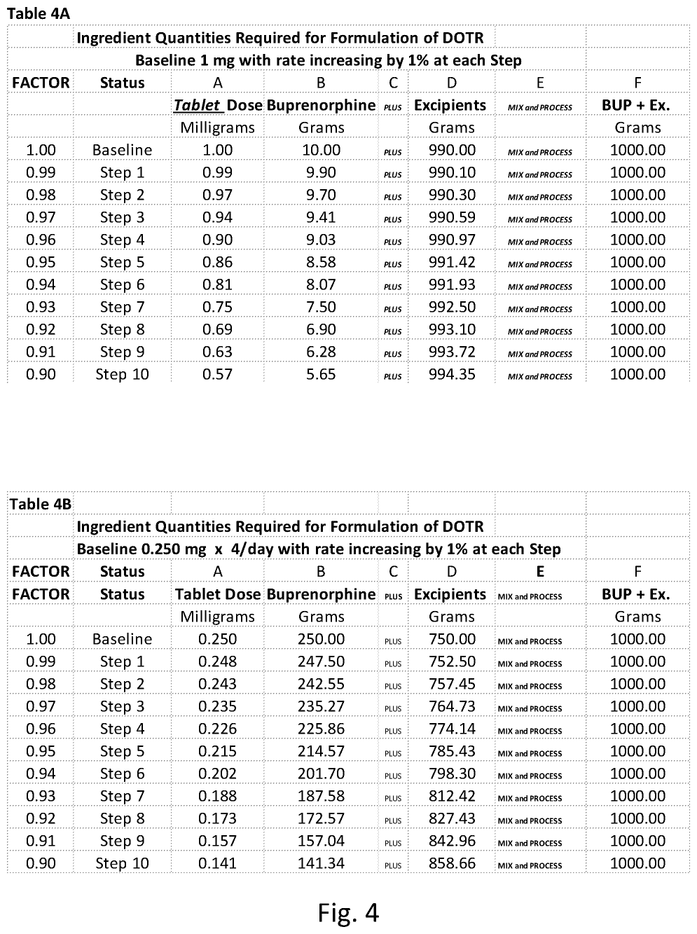

FIG. 4. Table 4A is titled, “Ingredient Quantities Required for Formulation of DOTR, Baseline 1 mg with rate increasing by 1% at each Step.” Table 4A shows the amounts of buprenorphine and amounts of excipients required to make Diagnostic Opioid Taper Regimens for a DOTR that will enable administration of tablet with a dose of 1 milligram of buprenorphine.

Table 4B, titled, “Ingredient Quantities Required for Formulation of DOTR Baseline 0.250 mg×4/day with rate increasing by 1% at each Step” shows the amounts of buprenorphine and excipients to make Diagnostic Opioid Taper Regimens for a DOTR intended for administration of four tablets per day with each tablet being 250 mg.

Both Table 4A and Table 4B show Diagnostic Opioid Taper Regimens dosages decreasing at each Step by 1%. The clinical driver for divided dosages is a patient who is getting to lower dosage levels. At these lower dosage levels, the patient's mu-opioid receptor occupancy rate in nervous tissue will fall below the occupancy rate necessary to avoid symptoms of withdrawal unless dosing is more frequent than is found at the 24-hour dosing interval recommended by the manufacturer for one-dose-per-day dosing. The higher dosages, for example, 12 mg once per day, can be sufficient for a patient to maintain their mu-opioid receptor occupancy rate at a level sufficient to avoid symptoms of withdrawal. However, for the patient who has elected to taper, in following their plan, will want to take smaller dosages per day. The approach of dividing the daily dosage can be utilized to achieve this. When the divided dosages start in a patient's regimen is a topic for the patient and their provider to resolve. Because there are no commercial dosages at this time that would easily permit divided dosages, a patient at 2 mg/day could start by cutting a 2 mg Suboxone® film into quarters. Because the total daily dosage will be kept constant at 2 mg, small errors in the precision cutting would not be relevant, particularly in light of the generally accepted half-life of buprenorphine.

Tables 4A and 4B are organized by horizontal Rows showing Baseline and Steps 1 through 10, and then organized also by vertical Columns.

### Legend for Columns:

Column A shows the dosage in milligrams of the tablet that will be made using ingredients in Columns B and D on same Table Row as “Tablet Dose”.
Column B shows the quantity of buprenorphine that goes in mixing vessel to make that Step.
Column C shows the instruction to “mix” ingredients from Columns B and D.
Column D shows the quantity of excipients that goes in mixing vessel to make that Step.
Column E is the instruction to “mix and process” ingredients from Columns B and D
Column F shows the sum of Columns B and D, a computed sum, as a cross-check.

Column heading of “Factor” indicates the factor by which the previous Step's dosage is multiplied to yield current Step's dosage.

Column heading of “Status” shows point the patient is in the taper, e.g., Baseline or Step 1 through 10.

The Tables in FIG. 4 show the amounts of the ingredients of buprenorphine and excipients necessary for formulating approximately 10,000 tablets. The actual number will be slightly less than 10.000 due to trace amounts remaining on equipment. The methods of achieving total homogeneity when starting with relatively small percentages of active pharmaceutical ingredient are established and should be utilized.

For example, a total daily dosage of 1.000 milligram in a DOTR that is reducing by 1% from Baseline to Step 1 goes from 1.000 mg to 0.990 mg (1.000-0.010=0.0990). When that total daily dosage is being administered as four divided dosages of 0.250, as can be seen from Tables 4A and B, the divided dose goes from 0.250 mg at Baseline to 0.248 (actual 0.2475) mg in Step 1. The structure of Table B is the same as the structure of Table A. More detail on making the dosages and about issues of formulating with this level of precision and on making tapers starting at various dosages and reducing at various rates of tapering can be made by following instructions in section on “Method of making the invention.”

### DETAILED DESCRIPTION OF THE INVENTION

The invention comprises a sequence of slowly decreasing dosages of medication. The dosages decrease in a specific manner, in which the rate of decrease is precisely identified. Each dosage has a precise relationship to the previous and to the following dosage that is created as a result of arithmetic calculation and precision formulation when the regimen is manufactured. Much of this application is devoted to describing the arithmetic relationships these dosages have to the sequence. The clinical relevance is explained.

This invention is intended for use by patients in maintenance programs. The invention is a method of determining a clinically sound course of treatment for patients in medication maintenance programs who wish to reduce, or stop, opioid use. A medication maintenance program, such as an opioid maintenance program, aims to treat a patient with medication so the patient does not experience craving for opioids.

There is a broad range of patients in maintenance programs. Some will take several years to stabilize in their maintenance programs. At the same time, some patients will be ready to reduce their level of opioid consumption.

In this invention, the rate of dosage reduction grows larger (i.e., is more rapid) each time in the sequential regimen that the dosage is reduced. This increasing rate of dosage reduction—i.e., accelerating rate of dosage reduction—allows the patient to experience a sequence of increasingly rapid dosage reductions. For example, reducing the dose by 3% at one decrement and by 4% at the next decrement.

The name of the invention is Diagnostic Opioid Taper Regimen. This regimen is designed to enable a health care provider, working closely with a patient, to determine a course of treatment for that patient using the medication in the regimen. “Course of treatment” here means the dosages the patient will take over time including the amounts of reduction and the time intervals between dosage reductions. The regimen comprises medication intended to be utilized in specific dosages and at specific times. The regimen starts at a specific total daily dosage and remains at that dosage for a specified period of time, e.g., for several weeks. After specified intervals of time, the medication dosage decreases in a specified manner, by some percentage of said dosage.

As the amount of opioid in the medication regimen reduces, a patient will have a specific sequence of responses. When the regimen is used as directed by a health care provider trained in use of the regimen, a patient's response to the regimen may be meaningfully evaluated. The evaluation of the patient's response by the provider is intended to indicate an effective course of treatment with the medication utilized in the regimen. “An effective course of treatment” here means that the Diagnostic Opioid Taper Regimen indicates a realistic rate of dosage reduction that specific patient is able to comfortably tolerate currently.

The active ingredient in the regimen is the medication upon which a patient is dependent. At this point in product development, this active pharmaceutical ingredient intended for use in the invention is buprenorphine. Use with other medications is likely to be feasible and application likely to be broad.

A significant number of patients who are taking opioid medication wish to reduce or stop their use of opioid medication. Buprenorphine is commonly used to treat patients who have a dependency upon opioids by the mechanism of stopping the sensations of craving for opioids. Most patients taking buprenorphine are doing so as part of a medication maintenance program.

The regimen: The regimen starts with a period of a Baseline dosage and is followed by a series of Steps. The duration of the Baseline and the Steps all being of equal time. A Step is a period of some weeks duration, e.g., four weeks, during which all dosages consumed by the patient are the same dosage amount. This invention enables patients to undergo a series of dosage reductions as part of the diagnostic process, over a period of some months as the patient goes through a series of Steps, each with a different dosage. The rate of the dosage reduction increases in magnitude from one Step to the next Step. For example, after a few months, the velocity of the dosage reduction at some point will increase from 5% at the start of a Step to 6% at the start of the following Step. When the velocity of dosage reduction increases from 5% to 6%, the velocity of dosage reduction is accelerating by 1%. This increasing rate of dosage reduction is a key novelty in this invention. While at any one step, the acceleration may be modest, it is not long before the patient is undergoing Steps of a significant magnitude of dosage reduction. At some point, the rate of reduction will exceed the rate the patient can tolerate comfortably.

As the sequence proceeds from one Step of the regimen to the next Step, the milligram amount of the dosage decreases in a specified manner at the start of each new Step and the total daily dosage of buprenorphine is reduced as the patient proceeds from one four-week Step to the next four-week Step.

A slow and gradual reduction of a medication is called a “taper.” This diagnostic taper is designed so that at some point in the taper, essentially all patients will start to experience the sensation of opioid withdrawal, due to the controlled and continuous reduction of their prescribed opioid medication. This diagnostic opioid taper is specifically designed to induce a mild sensation of opioid withdrawal at some point during its course of administration. This sensation of opioid withdrawal experienced by the patient is then used as a “signal.” The patient is aware that the withdrawal “signal” is to be reported to their provider in a timely manner. The nature of withdrawal, and how it is characterized in the field of addiction medicine, is described below.

The rate of reduction of the diagnostic taper is intended to be relatively slow and designed with the intent that such onset of withdrawal is mild, and yet detectable at an early stage. “Relatively slow” here means that the clinician selects a Diagnostic Opioid Taper Regimen that goes forward at a rate that the clinician believes, based on the patient's history, would be a rational choice to elicit the information needed to determine the patient's course of treatment.

Broad range of patients: There is a broad range of histories among patients who enter treatment for widely varying degrees of opioid dependency. This translates into the need for treatments that go forward at different rates of dosage reduction. For example, patients might be candidates to reduce their dosages at rates of 3% or 5% or 10% per month, or at rates faster or slower than those. Recently published research finds that patients entering opioid maintenance programs after dependency on fentanyl are six percent more likely to be retained in maintenance programs with higher buprenorphine dosages, 16 mg/day vs. 24 mg/day. (Chambers, et al. 2023) That is, the higher the dosage, the greater the probability the patient will be retained in maintenance treatment. Very little is known about the capacity of patients at these relatively high daily dosages to taper.

Intended for stabilized patients: Essentially all patients who are considered candidates for this diagnostic taper will be patients already in treatment in a medication maintenance program, such as an opioid maintenance program. Essentially all patients who will be considered for this Diagnostic Opioid Taper Regimen will have stabilized at a dosage level seen by their provider as a reasonable level from which to start a tapering regimen. “Stabilized” here means that a patient has been taking the same dosage of medication, such as 2 mg per day of buprenorphine, for at least some minimum period of time, such as eight weeks or more, and there has not been the need to change that patient's prescription dosage during that time, nor has the patient reported changes in their dosage consumption. A provider will consider the duration of the patient's dependency and the intensity of their prior opioid usage in selecting a specific Diagnostic Opioid Taper Regimen. Different Diagnostic Opioid Taper Regimens start at different dosage levels and go forward at different rates of dosage reduction.

Clarification of terms: throughout this application there is reference to increasing the rate at which dosage reduction is occurring. This refers to increasing the percentage amount by which dosages are reduced as the sequence of dosages proceeds through time. For example, if the first three dosages in a sequence are reduced at some interval by 1%, 2% and 3% respectively, then at each decrease, the rate of that percentage decrease has grown larger (has increased) by 1%. The rate of dosage decrease is increasing. The rate of decrease is occurring at an increasingly rapid rate, i.e., the rate of decrease is accelerating. This increasing rate of decrease is at the center of this invention, both in concept and in embodiment, and is novel. Whether obvious or nonobvious, this idea of an accelerating rate of taper decrease in a diagnostic, or any other, regimen, has never been written of, nor discussed nor made into an embodiment to best of applicant's knowledge.

Range of Diagnostic Opioid Taper Regimens: Essentially all patients who are candidates for the DOTR will currently be in an opioid medication maintenance program or some form of stable and ongoing treatment with buprenorphine. Prior to involvement in maintenance programs, or the equivalent, some patients will have had a few months experience with opioid dependency, perhaps following prescription opioid medication after surgery. Other patients will range up to those taking medication for 10 years, or more, and in treatment for chronic pain from injury. Some patients will have been injection drug users, with very varying periods of opioid exposure, from months to decades. Some patients may be those exposed to opioid use as a result of the influx of fentanyl that is a component of many drugs sold illicitly in the U.S.

A provider will choose the Diagnostic Opioid Treatment Regimen with the most suitable rates of dosage reduction. For example, for a patient with long-term exposure to relatively high doses of opioid, the clinician would be more likely to select a Diagnostic Opioid Treatment Regimen in which the rate of taper reduction increases rather slowly. For example, the rate of dosage reduction might increase from one Step to the next from a 4% dosage reduction to a 5% reduction to a 6% reduction with the rate of reduction thereby increasing by 1% additional at the start of each new Step.

Potential confounding aspects to diagnosing: The rate at which a patient would be able to taper will be unknown prior to administration of the DOTR. A patient might, for a number of reasons, be unable to taper past a certain milligram/day dosage, due to a condition not related to the velocity of the taper. This possibility complicates the diagnostic situation because the patient might be able to taper to some degree and then reach what amounts to a physiological “set point” past which it is not possible for that particular patient to further taper without a significant degree of discomfort. “Discomfort” might describe the patient's reaction at some initial points in time but this description might well change to some form of a sensation that is essentially intolerable if a patient is physiological unable to taper, regardless of how slow the taper may be.

DOTR serves to reduce confounding factor of patient “set points”: The following accepts the idea that some patients, after a significant period of exposure to opioids, undergo what amounts to permanent physiological changes that make it very difficult, or not possible, to return to their pre-opioid-exposure physiological status. For some of these patients, it is not possible to function normally without consumption of some level of opioid. These patients are not candidates for tapering. Best practice requires that patients who are not candidates for tapering not be prescribed a taper.

The DOTR may have the capacity to reduce confusion regarding diagnosis. One central feature that is built into the DOTR design is the reduction of a patient's opioid dosage at a sufficiently slow rate such as to reduce the possibility of confounding these two interpretations, A and B, of a taper:
A) a taper that is reducing a patient's dosage too rapidly, that is, reducing at a velocity that exceeds the capacity the patient has to reduce their dosage, with;
B) a taper that has reduced a patient's dosage below the absolute level of opioid dosage that patient can tolerate, i.e., dropping below a patient's “set point”, in a way that has nothing to do with the rate of the taper.

The DOTR starts very slowly. This facilitates distinguishing between the possibilities that, 1) a patient is able only to do a very slow taper, and; 2) a patient is unable to taper, that is, unable to reduce the dosage level to any degree. For example, the probability of a taper being too rapid when going forward at 1% per four weeks, from the starting dosage (Baseline) to Step 1, is a relatively small probability, though not zero. If a patient were to start to experience symptoms of withdrawal with a taper of one percent per four weeks, it would raise the question about that patient's capacity to undergo any dosage reduction whatever. That would then indicate the need for careful exploration by the patient's health care provider.

The patient reporting symptoms of withdrawal can happen at any point during a taper, and it is possible that the symptoms at any time may be related to having: a) passed a physiological set point of that patient as opposed to; b) having reached the point in the DOTR in which the taper administered is too rapid.

Example of DOTR with a patient with a low level of opioid exposure: Consider a patient who had previously not been exposed to any significant degree of opioid medication. For example, consider a patient who had surgery a month ago, and may have developed a dependency over a post-surgical period of three to four weeks of pain medication. This relatively short-exposure patient would likely require a different Diagnostic Opioid Taper Regimen than a patient with a long history of exposure to high opioid dosages, for example, an injection drug user with two decades exposure. With a patient with a brief opioid history, the provider might choose a somewhat more rapid Diagnostic Opioid Taper Regimen. For example, the provider might select a Diagnostic Opioid Taper Regimen that reduces from a dosage by 2% in the first four weeks and then by 4% at the start of the next 4-week Step and then by 6% in the next 4-week Step.

The broad range of patient histories and patient tapering requirements is the reason that Diagnostic Opioid Taper Diagnostic tapers will be produced in a variety of regimens in which both the starting dosage can be varied, and the rate of dosage reduction can be selected, based upon patient history.

By monitoring a patient's response to the specifically selected DOTR, a provider obtains information about a patient's capacity to tolerate opioid dosage reduction; specifically, this is accomplished by knowing when the symptoms of withdrawal emerge. When a provider knows a patient's capacity to tolerate the ongoing rate of opioid dosage reduction, as it is happening, this allows the provider to determine a clinically sound course of treatment for that patient. The basis for the clinically sound rate of tapering is based largely on the patient demonstrating that a several rates of tapering did not produce symptoms of withdrawal. The patient and provider would be likely to choose one of the rates of tapering that did not produce symptoms of withdrawal as the rate of going forward with the patient's opioid taper. Such treatment would include opioid dosage reduction in those cases in which the patient wishes to reduce and is diagnosed as likely to benefit from dosage reduction. Facilitating such treatment is the main function of the DOTR, accomplished by identifying a suitable rate of tapering.

Word Usage and Terminology: Throughout this document, “Step” and “Steps” have been capitalized to emphasize their specific definitions and usage within this application. Within this document: “Step” refers to the duration of time during which a specific dosage level of medication per day—an unchanging amount of active agent—is prescribed and administered or self-administered. For example, in the phrase, “During Step 1, the total daily dosage is 0.99 mg,” the word “Step” refers to the entire duration of time of that Step, for example, 4 weeks, from the first dose of that dosage level until the last dose at that dosage level of 0.99 mg. In all graphs herein, a Step is shown by the plot of dosage amount being horizontal, that is, the y-value, buprenorphine, remains constant over time throughout a Step.

The phrase “Diagnostic Opioid Taper Regimen” uses capitalized words to indicate that this Diagnostic Opioid Taper Regimen, of this patent application, is a unique entity. This unique quality is supported by a Google search engine search for the term, in quotes, “Diagnostic Opioid Taper Regimen” that found no use (zero use) of this phrase, with or without capital letters, on the internet as of Sep. 20, 2023. Likewise, no usage (zero use) of “Diagnostic Opioid Taper” was found in Google search on Sep. 20, 2023. Nevertheless, the phrase “diagnostic opioid taper” and “diagnostic opioid taper regimen” have independent meanings, separate from any proprietary use of “Diagnostic Opioid Taper Regimen”, by referring to the general concept.

The verb “to administer” in the context of medication discussed herein refers to the prescribing and providing of a medication to a patient with the intent, understanding, and assumption that the patient will consume said medication. To “self-administer” medication means, herein, the patient has on their own, as when taking a tablet at home, consumes medication in the prescribed manner. Sublingual medication refers to medication intended to be placed under the tongue until absorbed.

The term “dosage” is understood to mean a quantity of medication. Nevertheless, the term “dosage amount” or “dosage level” is at times used when the single word, “dosage” might suffice.

Terminology used to describe symptoms of withdrawal and tapering: This patent application provides a detailed look at the way in which opioid tapers are often described. This detailed look is intended to provide a more complete understanding of terms often used in a generalized manner, such as a taper is “too rapid.” Also explained below is a key factor in all tapers: the patients' subjective experience of opioid dosage reduction. A level of anxiety or of depression that is intolerable for one person, might be a degree of anxiety or depression that would be tolerable for a longer period of time by another patient. “Intolerable” symptom of withdrawal here means that a feeling arises in a person such that the person immediately seeks to change the sensation of withdrawal, and as a first choice, would often choose consuming an increased level of opioid medication immediately in order to stop the sensations of withdrawal. Communication between patient and provider is key to dosage adjustments that often need to be prescribed in a timely and prompt manner if significant reversion to previous behaviors is to be interrupted.

Evaluating a patient's state of withdrawal: The DOTR revolves around a patient's subjective and objective experience of withdrawal symptoms. There are questionnaires that are used to measure a patient's status regarding withdrawal. These questionnaires supplement a health care provider's observation and understanding of their patient. Two sets of questions currently in use are the Clinical Opiate Withdrawal Scale (COWS) and the Subjective Opiate Withdrawal Scale (SOWS).

The Clinical Opiate Withdrawal Scale (COWS) records physical symptoms, such as pulse rate, pupil size, runny nose, tremor, yawning and restlessness. As regards “anxiety or irritability”, the COWS provides a scale increasing from “none” to “patient reports increasing irritability or anxiousness” to “patient obviously irritable or anxious” to “patient so irritable or anxious that participation in the assessment is difficult.” Numerical values are assigned to the selected answer and totaled. The extent of withdrawal is then rated from “mild” to “moderate” to “moderately severe” to “severe” based upon the totaled score.

The Subjective Opioid Withdrawal Scale (SOWS) is answered by the patient and then scored by the provider. The patient is asked to answer “yes” or “no” to questions such as, “I feel anxious” and to identify the presence or absence of physical symptoms, such as yawning, perspiring, eyes tearing, having goosebumps, shaking, having hot or cold flashes, and bone and muscle pain. Restlessness, nausea, stomach cramps, muscle twitching are rated. The final response request is to the phrase, “I feel like using now”, meaning, “I feel like taking an opioid at this time.” In neither the COWS or SOWS is depression a parameter that is evaluated nor is there an attempt to evaluate suicidal ideation or thought processes. The COWS and SOWS can be administered at intervals to track trends in the patient's status.

The Central Clinical Element of the Diagnostic Opioid Taper Regimen: The emergence of the first onset of relatively mild withdrawal symptoms in the patient who is prescribed this Diagnostic Opioid Taper Regimen is intended to provide the critical diagnostic signal to the patient and the provider. This signal, of the first withdrawal symptoms, is the indication that the patient may be reaching the maximum velocity of dosage reduction in the Diagnostic Opioid Taper Regimen that the patient is capable of tolerating. For example, if a few days prior to the emergence of the first symptoms of withdrawal, the patient started the Step in which the reduction was 6% from the previous Step's dosage, it could mean that the patient may have exceeded the maximum rate of opioid dosage reduction of which that patient is capable. It could be a fluctuation due to the other variables that can impact withdrawal sensations, especially if a person is approaching their maximum tapering rate. These factors can include fatigue, diet, alcohol consumption, consumption of other medications, consumption of illegal drugs, travel, stress, exercise, sleep deprivation and other factors. Close communication between patient and provider is key. At that point the patient and the provider discuss what would make sense in terms of the rate of a taper it could be prescribed. It would be logical, as some degree of withdrawal emerges, for the patient and provider to review a recent rate of tapering in the Diagnostic Opioid Taper Regimen that did not produce symptoms of withdrawal.

With the DOTR, the patient will have recently experienced rates of dosage reduction that did not cause symptoms of withdrawal. The Step immediately prior to the 6% dosage reduction had a 5% dosage reduction. The Step immediately prior to the 5% dosage reduction was Step with a 4% dosage reduction. For this to be meaningful, in terms of dispensing tapers with a rate of 4% or 5% per four weeks, it will be necessary for tapers of 4% and 5% per 4 weeks be available commercially. No such opioid tapering regimens are currently available, and significant changes in the formularies available to dispense at pharmacies would be necessary for the DOTR to have maximum impact. The lack of available tapers commercially needs to be considered as possibly contributing the relatively low level of successful tapering that is reported currently.

Categorization of patients by rate of taper: Educational and instructional materials produced by the major U.S. governmental health agencies that describe opioid tapers categorize patients by the rate of opioid taper that a patient can comfortably tolerate. For example, some patients can tolerate 5% dosage reduction of opioid per month while other patients can tolerate 10% per month. The Opioid Taper Decision Tool of the Veterans Health Administration (VHA) presents tapers from 2% reduction of current dosage per eight weeks, up to 20% per month, and tapers even more rapid. Materials from the U.S. Department of Health and Human Services (HHS) and the Centers for Disease Control and Prevention (CDC) also discuss opioid tapers in the same terms of the percentage reduction of current dosage per unit of time.

Active Pharmaceutical Ingredient in this Invention: Buprenorphine is the active pharmaceutical ingredient for which this patent is primarily designed. Buprenorphine is a partial opioid agonist. It is widely used in opioid maintenance programs. An opioid maintenance program provides patients with a level of opioid medication intended to prevent the emergence of symptoms of withdrawal. Opioid maintenance programs can also rely on behavior therapy, psychotherapy, medical and social support to improve outcomes. Buprenorphine exerts many of the effects of a full opioid agonist but is significantly less likely to cause a fatal overdose than a full opioid agonist. Patients usually report that it is difficult to stop the use of buprenorphine when in maintenance treatment. The consensus approved method of stopping opioids, including buprenorphine, is to slowly reduce the dosage over a period of time. Abrupt reduction of opioid medication is not a best practice approach to opioid cessation. The approach of a diagnostic taper regimen utilizing the accelerating rate of tapering described here could likely be applied successfully, from a physiological perspective, using another opioid, such as methadone, or utilized with another medication with which dependency can occur. Medication potency considerations and half-life considerations will affect dosage amounts, in terms of milligram calculations.

Intended use of this invention: This invention is intended for use in the field of addiction medicine. It is an innovative method of determining a clinically sound course of dosage reduction for patients and providers. There is no existing diagnostic opioid taper regimen that is produced commercially, and applicant is not aware of any experimental use. The concept of a diagnostic opioid taper regimen in which dosage reduction accelerates is believed to appear for this first time in this patent application and there is no awareness by this applicant of prior discussion.

Risk Assessment Necessary: Tapering is a process for which best practices requires a risk/benefit assessment of a patient prior to prescribing a taper. If the assessment concludes the probability of benefit of the opioid taper outweighs risk to that patient, this would be taken as an indication the patient may be a good candidate for a taper. The correct use of the invention requires both: 1) the skills of an appropriately educated health care practitioner, and; 2) a patient of at least average motivation to reduce, or stop, their opioid use, and; who has demonstrated the capacity to benefit from an opioid tapering program, and; has the capacity, or potential, for effective communication with their health care provider.

National Institute on Drug Abuse Research Priority: As noted above, in “Background,” this application for Diagnostic Opioid Taper Regimen is for an invention designed to “allow an assessment of the treatment trajectory in patients under treatment for SUD [substance use disorder] and related conditions that are considered important to the mission of NIDA.” The 2022 Omnibus Solicitation of NIDA notes this research priority and the Diagnostic Opioid Taper Regimen is closely aligned with this NIDA

### PRIORITY

Determining the Treatment Trajectory: The clinically advisable rate of tapering that is determined by the DOTR is a rate of tapering that can be accomplished by the patient without the emergence of symptoms of withdrawal that are sufficiently unpleasant so as to cause the patient to want to stop tapering. When a patient in maintenance treatment wants to stop tapering, best practice calls for providing the patient with a dosage level that stops the emerging sensations of withdrawal, often returning to the previous maintenance dosage. Unpleasant symptoms can cause a patient to abandon their opioid medication treatment due to significant levels of discomfort. The invention seeks to determine a tapering rate that is not unpleasant and at the same time accomplishes a taper within a timeframe that is acceptable to patient and provider. Whatever levels of discomfort do exist must be those that can be tolerated for the months or years a taper may require. The DOTR is administered with the assumption that some form of taper will be administered, following the DOTR. This means the long-lasting levels of discomfort must be relatively quite minimal, or not present, as a significant component of the taper prescribed following the DOTR. Levels of discomfort that do emerge will require timely, careful, ongoing, and successful management when favorable outcomes are desired.

The invention functions in this manner: A patient in an opioid medication maintenance program stabilizes at a constant dosage of buprenorphine for a minimum of eight weeks and is assessed as a good candidate for a taper. Then, over a period of time, weeks to months, the patient consumes a prescribed sequence of buprenorphine doses that comprise the herein described “Diagnostic Opioid Taper Regimen” (DOTR). During the sequence of buprenorphine doses, the dosage of buprenorphine decreases in an accelerating, stepwise manner. A “stepwise” manner here means that when the dosage of the medication is reduced, that the dosage for a fixed period of time remains at that new dosage level, i.e., at the new Step level. The recommended period herein at which the dosage remains constant is four weeks for each Step, adjustable at provider or researcher discretion, should adequate clinical factors be present.

The issue of divided doses: Tapering that involves relatively low dosage ranges, such as below 2 mg, requires divided doses. Sublingually administered buprenorphine normally peaks in the patient's blood serum within approximately two hours, or less, after consumption. Moreover, the blood serum levels necessary to maintain mu-opioid receptors in the nervous system at an adequately occupied level often cannot be maintained by once-a-day dosing in the 2 mg dose/day range. (“Adequately occupied” mu-opioid receptors here means mu-opioid receptors occupied at a rate sufficient to prevent the emergence of symptoms of withdrawal.) Therefore, to maintain adequate levels of mu-opioid receptor level occupancy, dosing is very likely to be needed several times per day for patients who are entering into the lower dosage (below 2 mg/day) ranges. The dosage now prescribed for once-a-day dosing for buprenorphine maintenance is approximately 12 mg/day, or more. With the 12 mg/day dosage in mind, divided doses could be needed at any level below the 12 mg/day buprenorphine.

For example, this means a total daily dosage of one mg of buprenorphine might be prescribed and consumed as either one-half mg twice each day or one-third mg, three times per day or, for some patients, one-quarter mg, four times per day. These amounts would currently be “off-label” prescriptions because dividing tablets is not approved, even as divided doses is a common practice.

Patient compliance with divided doses is likely to be an issue for discussion. However, an opioid prescribed 4× day is different than, e.g., an antibiotic prescribed 4× day. It is the nature of opioids that if a patient misses a dose, a symptom will emerge to remind the patient of their medication regimen. Even if a patient is somewhat irregular in self-administering their divided doses, if the patient consumes the total daily dosage of buprenorphine within the intended 24 hours, mu-occupancy is likely to be adequately maintained, over-all. Please see FIG. 4B for examples of divided doses. As discussed above, divided doses become increasingly necessary at low dosages because at low serum buprenorphine dosages the mu-opioid receptor occupancy rate in nervous tissue falls below the level necessary to prevent the emergence of symptoms of withdrawal. At daily dosages such as 12 (twelve) mg or 16 (sixteen) mg/day, it is usually possible to maintain the necessary level of mu-opioid occupancy with once-a-day dosing.

Symptoms of Withdrawal Used as a Signal: At the point in the Diagnostic Opioid Taper Regimen sequence at which the patient begins to first experience significant (as described herein) symptoms of opioid withdrawal, a detailed and specific treatment plan agreed upon by provider and patient is activated, either by pre-arrangement or as the symptoms present. Pre-arrangement of what trajectory of dosing will be selected when significant withdrawal symptoms present, requires careful planning and trust, but offers advantages, such as less chance that regression will occur due to patient anxiety. Pre-arrangement can help avoid the type of issues that arise when a provider is not available, e.g., late at night. When symptoms of withdrawal emerge, a patient may have an emotionally amplified response based on concerns that symptoms may become worse. This situation requires either urgent attention or implementation of a pre-arranged plan.

As noted herein at several points, a central component of the diagnostic aspect of the Diagnostic Opioid Taper Regimen is the patient being alert for, and then reporting, the emergence of the first symptom of withdrawal that may be indicating the Diagnostic Opioid Taper Regimen has reached a point that is too rapid for a specific patient.

When the signal of the first significant withdrawal symptoms occurs:
1) The patient informs their provider that symptoms of withdrawal are starting to emerge. A provider's system of communication within their office must be configured for this level of timely communicationffff. Either there is, as noted, a pre-set plan of action, or;
2) As the symptoms emerge, the provider and patient discuss the current level of medication and the most recent rates of dosage reduction that were administered, prior to the emergence of current symptoms of withdrawal. Provider and patient review how patient experienced comfort level at previous rates of dosage reduction.
3) The patient and provider decide on a relatively recent rate of dosage reduction at which there were not symptoms of withdrawal.
4) Patient and provider agree upon the rate of tapering at which the patient will go forward. This would logically be a recent rate of dosage reduction but not necessarily the most rapid rate of the DOTR that the patient has experienced prior to the current level at which symptoms of withdrawal emerged. The example above, of a patient experiencing withdrawal symptoms while tapering at 6% per four-weeks, and thus choosing a 5% or 4% per four-weeks as their next step in long-term tapering, is provided.

In summary, regarding the emergence of the symptoms of withdrawal: There are several approaches possible, but the overarching principle is that the patient must feel secure that when the symptoms of withdrawal emerge there is a plan that provides adequate medication to resolve the symptoms within a relatively short period of time.

Manufacture: Methods for making the tapers that patients will consume following diagnosis of the rate of tapering necessary, are found in U.S. Pat. No. 11,253,512, held by this applicant, JLG.

In this example, as noted, the treatment plan could call for patient to continue at taper level of last symptom-free dose from Diagnostic Opioid Taper Regimen.

At the point in the DOTR when the patient then completes the four weeks at the symptom-free Step to which the patient returned, the DOTR is complete for that patient. At some future time, assuming success of the DOTR and acceptance of pre-packaged tapering regimens, pharmacies will dispense tapers that have a variety of rates. The continuation with a standardized taper is not a part of the diagnostic taper regimen, however.

The process of the DOTR: Through use of the Diagnostic Opioid Taper Regimen (DOTR), the patient has slowly reduced from their baseline dosage, at a rate of tapering that is slowly growing more rapid. As the rate of tapering increases, at each dosage reduction a larger percentage of the dose is eliminated. The faster the rate of tapering, the greater is the likelihood the patient will experience symptoms of withdrawal. This is a conclusion that aligns with the fundamental concept underlying HHS, CDC and VHA guidelines. The fundamental concept expressed by these agencies is that individuals have a maximum rate of tapering of which they are capable. As noted, the foundational concept of these agency guidelines, as applied to this Diagnostic Opioid Taper Regimen, is that when the rate of tapering with the DOTR exceeds the patient's capacity to tolerate dosage reduction, symptoms of withdrawal emerge.

Structure of the Diagnostic Opioid Taper Regimen (DOTR) and some Examples: As noted above, the Diagnostic Opioid Taper Regimen (DOTR) is designed to enable patients, who are currently receiving buprenorphine as part of their opioid medication maintenance programs, to initiate a diagnostic opioid taper. For example, patients can start their DOTR at various Baseline dosage levels, such as 2, 4, 8, or 12 milligrams (mg) per day of buprenorphine. Dosages of 2, 4, 8, or 12 mg/day are currently produced commercially and commonly prescribed in opioid maintenance programs. The example immediately below, of a patient starting a Diagnostic Opioid Taper Regimen at 1.00 mg, provides more detail on this last phase of the DOTR and the transition to treatment with a taper of a specific velocity.

DOTR of patient starting at 1.00 mg and reducing at a rate that accelerates by 1% at the start of each new Step: This is for a patient who has stabilized at 1 mg of buprenorphine for at least eight continuous weeks in a buprenorphine maintenance program. The patient is approved for tapering and wants to taper either to a lower dosage or, to opioid abstinence, that is, zero opioid usage.

Baseline: The patient starts the Diagnostic Opioid Taper Regimen at the same dosage that has been the patient's constant stabilized dosage for at least eight weeks in their maintenance program, i.e., 1 mg/day, and continues consuming 1 mg/day for four weeks at Baseline, almost certainly as divided doses, e.g., 250 mg, q.i.d. (four-times-a-day). (The first four weeks of the DOTR are called “Baseline.”) The Baseline medication is manufactured by the manufacturer of the entire DOTR. Thus, even as the dosage in Baseline represents no dosage change from the patient's maintenance dosage, having the medication source be the same as the entire DOTR helps to guard against manufacturer variability in amount or appearance. This sourcing helps prevent any small difference between manufacturers' products being responsible for any observed or reported withdrawal phenomena during the first dosage level of Baseline and helps assure consistency throughout the DOTR.

The following sequence is administered:
1) Baseline, and each Step, in this DOTR has a time duration of 4 weeks.
2) At the first dose of each Step, the total daily dosage is reduced by a percentage of the immediately previous Step.
3) Therefore, at the first dose of Step 1, the total daily dosage is reduced to 1% less than each total daily dosage of the immediately preceding Baseline period of 4 weeks. In FIG. 1A, Step 1 can be seen to be a consistent horizontal line for the duration of the Step, indicating that the variable y, buprenorphine dosage, does not vary during Step 1; i.e., the same reduced daily dosage level of the first dose in Step 1 is then continued in every dosage throughout the duration of Step 1. This pattern of repeating the same dosage continues throughout all Steps with every dose in a Step being the same, whether divided or not divided.
4) Thus, when the total daily dosage of Baseline is 1 mg, the total daily dosage of every day in Step 1, following the 1% reduction, will be 0.99 mg, a 1% decrease compared with every day during the previous four weeks of Baseline being a dosage of 1.00 mg. The arithmetic is 1.00 mg minus 1%, which equals, 1.00 minus 0.01 mg=0.99 mg. Achieving this with divided doses shown in FIG. 4, Table B.
5) Unless interrupted by the emergence of symptoms of withdrawal (see below), the pattern of accelerating the rate of the taper by 1% at each Step, i.e., 1%, 2%, 3%, 4% and so on to a 10% reduction (decrement), continues.

At the start of each Step, that is, at the first dose of Steps 1 through 10, inclusive, the amount of the total daily dosage decreases by a decrement that is 1% greater than at the previous decrement point. For example, in a DOTR starting at 1 mg Baseline and accelerating by 1% at each new Step:
At the start of Step 1, the dosage decreases by 1% of Baseline's 1.00 mg, resulting in a total daily dosage of 0.99 mg for all of Step 1;
At the start of Step 2, the dosage decreases by 2% of Step 1's 0.99 mg, resulting in a total daily dosage of 0.97 mg for all of Step 2;
At the start of Step 3, the dosage decreases by 3% of Step 2's 0.97 mg, resulting in a total daily dosage of 0.94 mg for all of Step 3;
At the start of Step 4, the dosage decreases by 4% of Step 3's 0.94 mg, resulting in a total daily dosage of 0.90 mg for all of Step 4;
At the start of Step 5, the dosage decreases by 5% of Step 4's 0.90 mg, resulting in a total daily dosage of 0.86 mg for all of Step 5;
At the start of Step 6, the dosage decreases by 6% of Step 5's 0.86 mg, resulting in a total daily dosage of 0.81 mg for all of Step 6;
At the start of Step 7, the dosage decreases by 7% of Step 6's 0.81 mg, resulting in a total daily dosage of 0.75 mg for all of Step 7;
At the start of Step 8, the dosage decreases by 8% of Step 7's 0.75 mg, resulting in a total daily dosage of 0.69 mg for all of Step 8;
At the start of Step 9, the dosage decreases by 9% of Step 8's 0.69 mg, resulting in a total daily dosage of 0.63 mg for all of Step 9;
At the start of Step 10, the dosage decreases by 10% of Step 9's 0.63 mg, resulting in a total daily dosage of 0.57 mg for all of Step 10.

The table immediately below is an overview of the Diagnostic Opioid Taper Regimen of the patient whose dosages are described immediately above who stabilized at a Baseline dosage of 1.00 mg of buprenorphine total daily dosage. See also FIG. 1A and FIG. 1B and Description of Drawings.

Below is table summarizing DOTR that accelerates by 1% at each Step. This recapitulates the above patient's regimen.
First Column: “STATUS,”: Signifies the dosage level at which the patient is within the DOTR.
Second Column: “% reduction” is the percentage reduction from the immediately previous Step.
Third Column: “Dosage”: Patient's dosage at that Step.

| STATUS | % reduction | DOSAGE mg |
|---|---|---|
| Baseline | 0% | 1.00 |
| Step 1 | 1% | 0.99 |
| Step 2 | 2% | 0.97 |
| Step 3 | 3% | 0.94 |
| Step 4 | 4% | 0.90 |
| Step 5 | 5% | 0.86 |
| Step 6 | 6% | 0.81 |
| Step 7 | 7% | 0.75 |
| Step 8 | 8% | 0.69 |
| Step 9 | 9% | 0.63 |
| Step 10 | 10% | 0.57 |

This pattern of a slowly accelerating rate of tapering, from 1% to 2% to 3% and up to 10% may be applied to a Diagnostic Opioid Taper Regimen at any starting dosage, e.g., 1, 2, 3, 4 mg, or any milligram amount. The basic concept is that the rate of tapering accelerates. This is a major feature of the invention. The acceleration permits the patient to experience, in a precisely ordered sequence, a series of rates of tapering. Monitoring the patient closely as the patient is exposed to this series of tapering rates is a key part of the concept on the method of the invention. No embodiment of this concept of acceleration of the tapering rate, applied to opioid tapering or tapering of medication nor of any related form of diagnostic endeavor, was found in prior art by this applicant. The general approach of trial-and-error to determine a tapering rate in which patients attempt to divide their own doses is minimally related and lacks the capacity to utilize the acceleration concept. The current method of trial-and-error is constrained by a lack of both accuracy and precision imposed, in part, by patients dividing their own medications.

Patients respond to the percentage decrease: In the examples shown in FIGS. 2A and 2B, the key idea in health agency material and in the Diagnostic Opioid Taper Regimen (DOTR) can be seen, i.e., reduction by percentage. Patients at any given dose respond to the percentage of the decrement and not the absolute milligram amount of the decrements. Whether the starting dose is 1, 2, 4, or 8 mg/day, when the reduction rate remains consistent, the sequences of dose reduction are proportionally the same. For example, assuming patient stability at each of these starting dosages, a 1% reduction from 1.00 mg to 0.99 mg has the same impact as a 1% reduction from 2.00 mg to 1.98 mg, or from 4.00 mg to 3.96 mg, and so on. In DOTR, reductions at each new step are done by percentages. The extent to which patients are responding to percentage decreases at very low dosages will benefit from further exploration.

Current Practice Compared with this Diagnostic Opioid Taper Regimen: Some of the patients who are currently assessed as being able to benefit from an opioid taper will elect to taper. With these patients, providers normally attempt to determine an optimal course of tapering. This involves trying a series of dosage reductions in order to determine what amount of reduction a patient can best tolerate. In other words, the concept has a resemblance to a diagnostic taper but lacks the necessary precision for clinically consistent and valid results. As noted above, the dosages that are manufactured commercially in the U.S. are 1, 2, 4, 8, 12, and 16 mg of buprenorphine. Indivior makes Suboxone® in 2, 4, 8, and 12 mg buprenorphine and Orexo makes Zubsolv® with doses of a stated bioavailability of 1, 2, 4, 8, 12, and 16 mg. Zubsolv® and Suboxone® are four parts buprenorphine to one part naloxone.

It is generally accepted in the field of addiction medicine that each patient has a maximum rate at which they can reduce their opioid medication and yet avoid the taper-disruptive symptoms of withdrawal. The commercially available dosages and formats make it relatively difficult for a patient in the low dosage range to mechanically divide their prescribed dosages in a way that would permit many patients to taper. For example, it is not a reasonable expectation that patients take a 2 mg dose obtained from the pharmacy and physically reduce it accurately and consistently by 10%. When considering that effective administration of buprenorphine often requires divided doses, it becomes even more unrealistic to expect patients to be capable of creating effective tapers. (Divided doses refers to the practice of dividing a total daily dosage and then consuming that same daily total of the medication in the several divided doses during the day.)

As noted above, with buprenorphine, it is likely necessary for most patients at lower dosage levels to divide the daily total dosage if a level of serum buprenorphine adequate to maintain sufficient mu-opioid receptor throughout a 24-hour period is to be maintained in nervous tissue. For those patients who are relatively less sensitive to opioid dosage reduction, and therefore require less precision in dividing their doses, it may be possible for some patients to divide their prescribed dosages and to achieve a workable taper. Official prescribing inserts advise against dividing doses. The number of patients who stop their opioid maintenance programs seems to indicate that the percentage of patients who manage to stop is relatively small, i.e., less than approximately 10 (ten) %.

Therefore, the situation described immediately above, i.e., lack of dosage amounts that can be used for an effective taper, is the situation faced by providers, and applies to trying to determine an optimal course of treatment for a patient with opioid dependency. This invention introduces a precision formulated sequence of dosages, the Diagnostic Opioid Taper Regimen, in which the amount and percentage of dosage reduction is reliably known. As detailed, patient and provider then use the DOTR to determine an effective trajectory of opioid reduction treatment. This availability of the DOTR will create a change from the current situation of patients and providers relying on a relatively imprecise trial-and-error approach. Currently this approach results predominantly from the absence of commercially available dosage sequences that allow for a gradual and quantitatively reliable reduction in a patient's opioid medication dosage.

Reducing a patient's dosage until some symptom is reported by a patient is accepted practice when seeking to determine a treatment trajectory. The current trial-and-error and DOTR approaches have a common goal: They both aim to achieve a specific outcome of identifying a taper rate by triggering a signal of withdrawal symptoms. However, the trial-and-error approach is so far less precise than the DOTR that to identify a specific rate of tapering, using trial-and-error, would probably not be possible in most cases. Part of the reason for seeking individualized taper rates is that if a very slow of “one-size-fits-all” approach that is tried that people who are capable of more rapid tapering will require much more time than their individual physiology might permit with an optimal taper rate.

Close cooperation and communication between patient and clinician is essential for the DOTR approach. The practice requires the provider, or a suitable team, to be continuously available and receptive to patient reports. The alternative to a 24/7 alertness approach is a pre-planned course of action. The pre-planned course of action provides the patient with instructions and the appropriate medication to use in the event withdrawal symptoms emerge when timely treatment not otherwise available. This approach of patients having a prescribed plan in advance, that can be put into effect when withdrawal symptoms emerge, requires a high level of trust being placed in the capacity of the patient.

Differences between the DOTR method and informal methods of determining opioid taper rates: The current approach to determining a patient's clinically sound rate of opioid tapering often involves having patients cut/divide their prescribed medication, tablets or films into pieces. The practice can, at some higher dosages, achieve tapers that are vaguely approximate arithmetically to an effective taper. Indeed, some patients appear to use such a method to taper effectively. The Diagnostic Opioid Taper Regimen, in contrast, achieves the dosage precision possible with commercial pharmaceutical production methods and thereby serves patients who are more sensitive to dosage reduction and require greater precision in determining an optimal rate of tapering. The DOTR level of precision is orders of magnitude more precise than the precision a patient with a pill cutter or a pair of scissors can achieve and based on current success rates would probably serve far more than the current approach.

The DOTR dosages are sufficiently precise to allow various specific rates of tapering, such as 2% per eight weeks or 5% or 10% per four weeks, to be diagnosed by the diagnostic opioid taper regimen. Enabling a clinically sound determination of consistent and specific rates of tapering is the central feature of the Diagnostic Opioid Taper Regimen.

This invention thereby introduces a practical, consistent, and long-sought method of determining a clinically sound rate of tapering for patients who wish to lower their opioid consumption. As a requirement for more than experimental use, efficacy determined through randomized controlled trials will be needed.

Various preconceptions hinder acceptance of need for small decrements: Conceptually, it can be difficult to accept that very small quantitative differences in dosage reductions can result in significantly different reactions among patients. For example, it is easier to accept that there will be significant symptoms of withdrawal with a patient who is reducing their daily dosage by one milligram, from 10 mg to 9 mg/day, than in a patient who reduces by one-tenth of a milligram from 1.0 mg to 0.9 mg/day, even as both reductions are a 10% reduction, and by some theories should result in similar degrees of symptoms of withdrawal.

The conclusion that emergence of withdrawal symptoms is related to the percentage of dosage reduction is drawn from generally accepted theories as expressed in materials produced by HHS, CDC and VHA. In these documents (please see agency sources in “References”) patients are categorized as being able to taper at specific rates, e.g., such as 10% per month or 5% per month. It is broadly accepted that some patients can tolerate a reduction of 5% per month but cannot tolerate a reduction of 10% per month. It can be difficult to accept that this arithmetic of tapering applies at very low dosages.

For example, it can be difficult to accept that a taper from 1.00 to 0.95 mg/day, i.e., five percent, will be possible for some patients to tolerate while for the same patient, reducing from 1.00 to 0.90 mg/day, i.e., ten percent, is not a taper that patient could accomplish without experiencing relatively severe withdrawal symptoms.

It is probable that dosages employing small decrements and formulated for research will be required to further clarify the current understanding of any causal relationship between rates of tapering and the points at which there is the emergence of withdrawal symptoms.

Use of the Diagnostic Opioid Taper in Context of Treatment: As noted, this invention is intended for patients who first have stabilized in an opioid maintenance program at a specific daily rate of opioid use—for example, a patient in an opioid maintenance program has stabilized at 1 or 2 mg per day of buprenorphine. Following stabilization in their maintenance program, items 1 through 4, immediately below, show the sequence of some of the treatment options indicated for this patient.
1) Patient has stabilized in opioid maintenance program at specific dosage for 8 weeks or more.
2) A suitable Diagnostic Opioid Taper Regimen (DOTR), described in detail in this application, is prescribed by provider.
3) Patient starts DOTR and continues DOTR until withdrawal “signal” received by patient and discussed with provider.
4) The clinically indicated rate of tapering using buprenorphine is determined, per the administration of the DOTR. Details of how this decision is reached regarding a specific rate are above in The Central Clinical Element of the Diagnostic Opioid Taper Regimen.
5) Based upon the results of the DOTR, the provider prescribes the rate of tapering that is determined to be best for that patient.
5) The patient self-administers buprenorphine as prescribed, based upon results of DOTR.

At this point, when the patient is starting to be prescribed the rate of tapering determined by the DOTR to be optimal or near optimal, there are several likely possibilities of how treatment will go forward: The possibilities for the patient at this point are:
A) Patient continues to taper at the rate that has been determined, following the DOTR, either to a desired dosage level or to opioid abstinence.
B) Patient tapers for some months at the newly prescribed rate of tapering and then starts to feel that the taper prescribed following the DOTR is inadequate to prevent emergence of symptoms of withdrawal.
C) Patient states within a few days of starting a newly prescribed taper that symptoms of withdrawal are again emerging or states that the rate of decline of the taper is too rapid.

Each of these outcomes immediately above, A, B, and C, calls for an intervention by a health care provider for whom these types of scenarios are routine. If the rate of tapering ultimately proves too rapid, the provider can either prescribe a taper at a slower rate or start a DOTR again at a dosage slightly greater than the dosage at which the patient started to show symptoms of discomfort. As noted, there are no commercial tapers that the provider could prescribe. The option of being able to prescribe a taper of the rate indicated by the DOTR that could actually be dispensed would require the availability of tapers at various rates being dispensed by pharmacies.

Duration of Steps in DOTR: One advantage for the patient of a diagnostic opioid taper in which the Steps are of four weeks duration (during which 4 weeks the level of medication does not change) is that the patient's system is given sufficient time to fully adjust to a dosage decrease. (In “Claims” a range of time durations for Steps is claimed.) If the rate of dosage decrease has been “too rapid,” symptoms of opioid withdrawal will emerge. Here, “too rapid” means, “the rate of dosage reduction produced symptoms of withdrawal.” A taper at a specific rate may need to be sustained over a period of months or years during which time the patient must be sufficiently comfortable for the taper to achieve its objectives.

Variability of DOTR rate of taper acceleration: The method of this Diagnostic Opioid Taper Regimen is to prescribe for the patient a series of tapering rates. To review: each successive Step in the tapering sequence represents tapering at a more rapid rate than the previous Step. For example, to review, from Baseline to Step 1 there is a 1% decrease in the daily dosage of buprenorphine. The decrease from Step 1 to Step 2 is a 2% decrease. The dosage decrease from Step 2 to Step 3 is a 3% decrease. The decrease from Step 3 to Step 4 is a taper of 4%. In this regimen, the rate of the decrease grows larger by 1% at each Step, as is shown in FIGS. 1A and 1B. The increase in velocity of the dosage decrease is a parameter that could be controlled by an investigator in a clinical trial. Should the Diagnostic Opioid Taper Regimen become available commercially, it is likely that DOTRs with a range of taper accelerations will be available. For example, FIGS. 3A and 3B show how the reduction in Steps 1 through 5 increases from 2%, to 4%, to 6%, to 8% to 10% and then five additional Steps in which there is a decrease of 10% at each Step. Comparing FIG. 1 and FIG. 3 rates of reduction shows the parameter of rate of reduction over the course of the DOTR as a variable. In “Claims,” a range of rates of accelerating dosage decrease are claimed. This approach of changing the rate of decrease as desired would permit serving a broader range of patients than with just one standardized DOTR. For example, the DOTR with the more rapid reduction would be suitable for patients who may have formed an opioid dependency from a relatively brief exposure to prescribed opioids, e.g., following recent surgery. Selection of the appropriate DOTR requires a provider skilled in addiction medicine.

As seen from foregoing, the hypothesis here is that by going through a series of gradually increasing rates of dosage decrease, it will be possible to determine a rate of tapering that ultimately proves slightly too rapid for the patient. The model used here is each Step of four weeks duration. As noted, experimentation may show there are advantages to Steps of duration shorter than four weeks or longer than four weeks. It is considered important to create a time duration for a Step that has a high probability of producing a result that leads to a sound course of tapering. A sound course of tapering is important because of the potential delays to treatment that will occur if a patient is given a taper slower than their capacity to taper or the disruptions that will occur if a taper is too rapid, especially should regression occur. “Regression” here means the patient returns to the unprescribed usage of opioids. It will be productive for patients and providers to remain mindful that even as this is a diagnostic taper, that tapering is happening as the diagnostic process goes forward; that is, time is being utilized productively.

Novel aspect: As noted above, one novelty of the Diagnostic Opioid Taper Regimen is the fact that the rate of tapering changes as the patient is administered a series of dosage Steps. With the DOTR the rate of the tapering in increasing, as dosages become smaller and smaller. Although no one has manufactured a treatment taper that decreases at a continuous specific percentage, it has been discussed and proposed as a form of treatment. Linear tapers have been proposed as a form of treatment. What has not been proposed nor discussed nor manufactured is a diagnostic opioid taper regimen in which the velocity of the taper continues to increase, such as in this patent application. This is therefore novel in concept and embodiment. As noted, because of the novelty, there is no prior art of a “diagnostic opioid taper” nor “diagnostic opioid taper regimen” and is why these phrases are not found in an internet search, or patent search.

To review: In FIG. 1A is shown a decrease in dosage amounts that becomes a larger decrease, by 1%, from Step-to-Step, with the 1% increase in taper velocity at each Step. In FIGS. 3A and 3B are shown the 2% additional velocity in taper rate from Steps 1 to 5, as seen from 2% to 4% to 6% to 8% to 10% and then the percentage rate of dosage decrease remaining constant at 10% while going from Steps 5, 6, 7, 8, and 9 respectively.

The rate of velocity change may be selected by the researcher or provider. This relates back to the fundamental way of speaking of patients in U.S. health agency materials from HHS, CDC, and VHA, that is, in terms of each patient having a maximum percentage rate of dosage reduction that is possible.

To summarize, as noted, a DOTR of a constant 1% increase in taper velocity from Step-to-Step, would be for patients with longer histories of opioid use. The regimen with five successive 2% increases in velocity of decrease and then 5 successive 10% increases in velocity of decrease is a more rapid decrease of dosages and exposes patients to a series of more rapid velocities. This latter approach would more likely be for patients with less severe exposure to opioids, possibly that of a few months, e.g., post-surgery. This increasing velocity of dosage decrease in a series of tapers, as noted, is a variable not previously identified in the literature nor embodied. The concept is not found in a diagnostic tool nor in a treatment regimen. It can be objectively regarded as novel both in general concept and in the specific ways in which the rate changes from Step-to-Step.

This Diagnostic Opioid Taper Regimen will allow categorization of patients with opioid use disorder by the rate of taper that is a sound clinical trajectory for a patient. In doing so, the DOTR is categorizing patients with SUDs into clinical subtypes. The Diagnostic Opioid Taper Regimen fills a gap in the diagnosis and treatment of opioid use disorder and opioid dependency. The DOTR holds promise for research efforts as well as tapering for other classes of medications such as benzodiazepines and SSRIs, pending demonstration of efficacy.

### The Method of Making the Invention

The invention is made by mixing buprenorphine with excipients, as detailed below. This process produces precision dosages in a specific sequence. The invention requires a sequence of eleven different dosages to be made for each version of the Diagnostic Opioid Taper Regimen (DOTR). Each dosage is of a smaller amount of active ingredient, buprenorphine, than the preceding dosage amount. The parameters of the starting dosage; rate of dosage change; total number of Steps, and; duration of each Step, can be varied.

Each dosage in the DOTR sequence reduces from the previous dosage at a slightly more rapid rate of tapering than the previous dosage decreased from its previous dosage. The rate of reduction is here measured as a percentage decrease. The larger the percentage decrease, the more rapid will be the rate of reduction.

For example, one embodiment of the Diagnostic Opioid Taper Regimen has each successive dosage reduction being 1% greater than the previous reduction. Baseline is the first dosage level in the DOTR formulation example provided in this Method of Making section. The Baseline is reduced by 1% and the first dosage of Step 1 is 1% less than the dosage level of Baseline. When a Step is of four weeks duration and there is one dose per day, then every dosage for that four weeks, all 28 doses, are the same dosage amount. When a Step is of four weeks duration and there are two divided doses per day, then each of those 56 doses are the same. In this example of formulation, after reducing Baseline by 1% to produce Step 1, then:

To determine the dosage for Step 2, reduce Step 1 by 2%.

To determine the dosage for Step 3, reduce Step 2 by 3%.

Thus, the rate of dosage decrease is therefore getting more rapid. This pattern continues, with Step 9 being reduced by 10% to create Step 10. Please see FIG. 4, and also narrative of FIG. 4, below.

Thus, the diagnostic taper proceeds from the first decrease at a rate of 1% going from Baseline to Step 1, to a rate of decrease from Step 9 to Step 10 of 10%. That is the rate of decrease has increased from 1% to 10% as the DOTR progressed from Baseline to Step 10.

Different versions of the DOTR can differ in several parameters:
1) The starting dosage;
2) The rate at which the rate of tapering increases from one Step to the next Step, and;
3) The number of doses each day, while maintaining total daily dosage the same.

Experimentation would be expected to explore changes in these three parameters with randomized clinical trials, for example, with different starting dosages and different rates of diagnostic taper acceleration. That is, the rate at which the rate of dosage reduction increases would be a focus of experimentation. The extent to which there are divided dosages would be another variable of experimental interest. All of these variations will be able to be addressed by changing the amounts of the ingredients so as to produce Diagnostic Opioid Taper Regimens with various parameters. The duration of the various Steps would also likely be a focus of experimentation. This research would attempt to determine what constitutes efficacious tapers across a broad variation of patients. To make these various DOTRs it is necessary to follow the manufacture protocol described in this section.

With the exception of the changes in the amount of ingredients at various steps, the method of making each Diagnostic Opioid Taper Regimen and the method of making every given dosage amount is generally the same. That is, buprenorphine and excipients are added together using the specific dosage amounts required and are then mixed to adequate homogeneity. Tablets are then produced from the mixture. Some of the factors of pharmaceutical dosage production that are normally involved in tablet production are noted below, in Formulation. Changes in the ratio of active pharmaceutical ingredient to excipients can affect parameters of formulation, such as mixing times.

The tables referenced in this Method of Making the Invention section and showing the amounts of the ingredients are in Drawings section and identified by FIGS. 4A and 4B.

The Tables are organized by Rows, showing the Baseline and the 10 Steps, with Steps 1 through 10, and by Columns, as described.

### Legend for Columns:

Column A shows the dosage of the tablet made from ingredients on that row.
Column B shows the quantity of buprenorphine that goes in mixing vessel to make tablets for that Step.
Column C says “plus”, meaning ingredients from Column's B and D are added together. Column D show the quantity of excipients that goes in mixing vessel to make the 100 mg gross weight tablets of that Step.
Column E is the instruction to “mix and process” the ingredient mixture resulting from adding together Columns B and D.
Column F shows the computed sum of Columns B and D, as a cross-check.
Column heading of “Factor” indicates the factor by which the previous Step's dosage is multiplied to yield current Step's dosage.
Column heading of “Status” shows point the patient is in the taper, e.g., Baseline or Step 1 through 10.

The tables show the amounts of the ingredients of buprenorphine and excipients required to formulate 10,000 tablets. Trace amounts will remain on equipment and actual production will be less than 10,000 but will not affect composition of each individual tablet. The methods of achieving total homogeneity when mixing relatively small percentages of active pharmaceutical agent are established and should be utilized.

Buprenorphine sublingual tablets are commercially produced in the U.S. in the general range of some of the products herein. There are 2 mg sublingual tablets of buprenorphine (100 mg gross weight tablet), Subutex®, (Indivior) There is a 0.7 mg Zubsolv® (Orexo) tablet with bioavailability of 1 mg.

Small differences between dosages to be formulated: Note the DOTR with divided doses that starts at a Baseline of 0.250 mg×4/day and in Step 1 is 0.248 mg×4/day. The degree of precision of successive dosages of 0.250 mg and 0.248 mg with a dosage decrement of 0.002 mg might currently be considered, by some, both unnecessary and unattainable. The arithmetic shows 0.2500 and next dosage, by calculation, is 0.2475 for a decrement of 0.0025 mg×4 per day equaling 0.01, i.e., one-hundredth mg/day. That is, as the patient goes from Baseline to Step 1, their net decrease per day will be 1/100 of a milligram per day.

This reduction is best viewed as part of a larger regimen that results in lowering the Baseline by 43% after nine Steps, even as it is reduced by only 1% in this first Step. As to being unattainable, the challenges of making dosages that differ by small amounts is addressed herein and in U.S. Pat. No. 11,253,512 B2 issued this applicant, JLG.

Here is one example of how to make the dosages. The DOTR described in FIGS. 1A and 1B, with title in FIG. 1A of “Diagnostic Opioid Taper Regimen, Baseline 1 mg, 1% to 10% Step decreases.” FIGS. 1A and 1B show a Baseline of 1 mg with rate of taper increasing by 1% at each Step is utilized. The information directly pertinent to formulating this DOTR is in FIG. 4, Table 4A, titled “Ingredient Quantities Required for Formulation of DOTR; Baseline 1 mg with rate increasing by 1% at each Step.” The protocol for formulation used is the same for all DOTRs, except that the amounts of buprenorphine and excipients vary and thus parameters, such as mixing times, may vary as ratios of ingredients change.

Directions for formulating a tablet with a gross weight of 100 milligrams containing 1.00 milligram dosage of active pharmaceutical ingredient buprenorphine: To make the first dosage (Baseline) in the DOTR that starts at 1 mg/day in Baseline and in which the rate of tapering accelerates by 1% at each Step, as shown in FIG. 4, Table 4A, do the following: Take 10 grams of buprenorphine and mix with 990.00 grams of excipients to total homogeneity, as described below. Remove 100 mg aliquot of the mixture to make a tablet that contains 1 mg of buprenorphine. Repeat, preferably with production equipment and procedure that allows for mass production of tablets. Makes approximately 10,000 tablets.

Formulating tablet having dosage of 0.99 milligram buprenorphine: To make the second dosage, that is, Step 1, in the DOTR that starts at 1 mg/day and in which the rate of tapering accelerates by 1% at each Step, as shown in FIGS. 1A and 1B, with formulation amounts in FIG. 4, Table 4A, do the following: Take 9.90 grams of buprenorphine, as shown on the same Row as the Tablet dose of 0.99 mg of Step 1 and mix to total homogeneity with 990.10 grams of excipients. Remove 100 mg aliquot of the mixture to make a tablet that contains 0.99 mg of buprenorphine. Makes approximately 10,000 tablets.

To complete this DOTR, i.e., to finish Steps 2 through 10, inclusive, follow the same general protocol used to make the 1.00 mg and 0.99 mg tablets so as to achieve each dosage. This will entail mixing the amounts in Columns B and D in the respective Rows of Step 2 through Step 10, in order to make the dosages, of: 0.97, 0.94; 0.90; 0.86; 0.81; 0.75; 0.69; 0.63, and; 0.57 milligrams of buprenorphine, respectively. Package using standard approved packaging protocols and labeling. Include instructional insert. Packaging identifies tablets by dosage and provides packaging that is designed to ensure consuming the medication in the appropriate sequence.

Diagnostic Opioid Taper Regimens with variations in key parameters: The above method can be applied to make Diagnostic Opioid Taper Regimens in which there is wide variability, as addressed in “Claims,” re: a) starting dosage; b) starting rate of taper; c) rate of taper change, and; d) divided doses. For example, Baseline, in practical terms, can start somewhere from 0.5 mg to 8.0 mg, inclusive. Starting rate of taper can be 0.5 to 2.0%. Rate of increasing change of taper from Step-to-Step can likely be 0.5% to 2.0%. This rate could be changed at some point in the taper, as well, at discretion of provider or researcher. Regimens using divided doses can be made, as described below.

Logical starting amounts of Baselines are 2, 4, and 8 mg because commercial dosages are available at 2, 4, 8 mg. This means that patients are more likely to have stabilized at these dosages in their maintenance programs. As research reveals more about comparative tapering rates, it will be better understood what rates of tapering acceleration from Step-to-Step results in more efficacious DOTRs.

As noted in detailed description of this invention, the lower the total daily dosage, the more likely that dosing at shorter time intervals becomes necessary, in order to maintain adequate opioid receptor occupancy. The overall method of pharmaceutical formulation remains the same for divided doses as the method described immediately above for formulating “Baseline 1 mg with rate increasing by 1% at each Step.” Ingredient quantities change relative to dosage being formulated and parameters such as mixing times can be affected.

Example of making two different DOTRs with same total daily dosage: Both DOTRs have a total daily dosage of 1 mg. However, one DOTR, A) is 1 mg once a day and the other DOTR, B) has four divided doses of 0.250 mg administered 4×per day. (1 mg×1/day=1 mg; 0.250 mg×4/day=1 mg.) For ingredients for DOTR “A,” see FIG. 4, Table 4A for “Baseline 1 mg with rate increasing by 1% at each Step” and for DOTR “B” see FIG. 4, Table 4B “Baseline 0.250 mg×4/day with rate increasing by 1% at each Step.” Table 4A shows the ingredients for once-a-day dosing and Table 4B shows ingredients for 4× per day dosing, resulting in same total daily dosage with 4A or 4B.

Follow the directions to make the dosage of any specific 100 mg (gross weight) tablet and mix the buprenorphine and excipients in the precise amounts specified. Follow the directions as provided above in the section on formulating a tablet with 1.00 milligram dosage. The amounts of buprenorphine and excipients are in the same respective columns of B and D as the dosage being formulated. The same demanding mixing protocols will be required.

Using this approach, of combining buprenorphine, the active pharmaceutical ingredient, and the various excipients selected for their specific qualities, any dosage desired can be formulated. More specifically, per this example: As shown in FIG. 4, Table A, the amounts produced through formulation of the sequence for DOTR if once-a-day dosing were possible, would be 1.00; 0.99; 0.97; 0.94; 0.90; 0.86; 0.81; 0.75; 0.69; 0.63, and; 0.57 milligrams. By following the instructions and ingredient amounts in FIG. 4, Table B, the amounts for the four-times-per-day dosages will be produced, that is: 0.250; 0.248; 0.243; 0.235; 0.226; 0.215; 0.202; 0.188; 0.173; 0.157, and; 0.141 milligrams. As noted, the total daily dosages will be the same, with the formulation guidelines for both A and B. The administration of the divided doses addresses the mu-opioid receptor occupancy rate.

Environment: The type and scale of formulation described in this “Method of Making the Invention” will generally require the environment of an advanced, well-equipped, commercial or academic laboratory. Some advanced compounding laboratories would be able to carry-out these directions. Advanced analytic equipment is required to confirm the dosages achieved in formulation work. Commercial-scale production could require larger starting quantities and equipment at scale. Specialized licenses for the laboratory and for personnel are required whenever buprenorphine is used in manufacture.

Formulation: Buprenorphine has poor oral bioavailability. Tablets are therefore formulated to permit sublingual administration. Naloxone is included in most U.S. formulations of buprenorphine in a 4 buprenorphine to 1 naloxone ratio to deter diversion. Some U.S. commercial products omit naloxone. The use of naloxone to deter has been questioned as to efficacy. Any of the Diagnostic Opioid Taper Regimens described here could be made with the 4 buprenorphine to 1 naloxone ratio, but from a strictly pharmaceutical efficacy perspective, naloxone is not an active ingredient in the tapering of opioids.

Excipients should be used that facilitate desired rate of production, including diluents. Excipients that promote pharmacological effectiveness should be utilized. Binders, lubricants and glidants should be used as needed. Uniformly combining small quantities of buprenorphine, or 4:1 buprenorphine/naloxone, into compressible powders will be a required step in the process, when such a product is desired. This poses a mixing challenge because of the relatively small quantities of buprenorphine and naloxone used and the high ratio of the other ingredients to buprenorphine and naloxone. Maintaining accuracy with dosages that are similar in milligram amounts requires ongoing attention. Tablets must be monitored for quality using standards established by the United States Pharmacopeia, including for content uniformity, breaking force, and friability as well as percentage and absolute content of the active pharmaceutical ingredient.

The invention claimed is:

1. A method of determining a clinically indicated rate of reducing the consumption of a substance upon which a person has a substance dependency, comprising:
a) specifying dosage amounts;
b) specifying durations of time for each dosage amount;
c) specifying rates of dosage reduction;
d) specifying dosage sequences, and;
e) administrating dosage sequences to persons diagnosed with a substance use disorder, substance dependency, or related conditions.

2. The method of claim 1, wherein the following parameters are varied within ranges of medication amounts and time durations, to produce an array of diagnostic sequences, such variable parameters comprising:
a) varying the starting dosages;
b) varying the duration of time during which a dosage remains constant prior to said dosage being reduced;
c) varying the rate of dosage reduction going forward in time, expressed as a percentage of the current dosage;
d) varying the rate of change of the rate of dosage reduction, so that from one dosage decrease to the next dosage decrease the rate of dosage reduction is accelerated, that is, the rate of dosage reduction is made to occur more rapidly;
e) employing these dosage sequences with any medications that can cause dependency;
f) employing these dosage sequences with opioids, benzodiazepines, and selective serotonin reuptake inhibitors;
g) using combinations of these parameters in a variety of configurations so as to create a broad range of diagnostic taper regimens.

3. The method of claim 1, wherein the first dosage of a regimen is in a range from approximately one-tenth milligram to twenty-four milligrams.

4. The method of claim 1, wherein the total daily dosage remains constant for each 24-hour day, for a period of one day to fifty-six days.

5. The method of claim 1, wherein the rate of dosage reduction from one dosage to the next is comprising:
a) a percentage of the current dosage amount, this reduction resulting in the reduction of the immediately following contiguous dosage amount;
b) the percentage reduction from one dosage to the next dosage ranging from a percentage reduction of about one-tenth of a percent reduction of the current dosage to a fifty percent reduction of the current dosage.

6. The method of claim 1, wherein the rate of the rate of dosage reduction is increased, comprising:
a) the percentage rate of dosage decrease from one dosage level to the next dosage level increasing by a finite percentage, and;
b) such percentage increase in the rate of percentage decrease ranging from about one-tenth of one percent to twenty percent;
c) the rate of dosage reduction being made greater from one dosage reduction to the next dosage reduction;
d) exposing the patient to a series of periods of time during which there are increasingly rapid rates of dosage reduction, i.e., the rate of dosage reduction growing more rapid from one time period to the next time period.

7. The method of claim 1, wherein the decreasing series of dosages is in sequence, with the percentage decreases at successive decreases being 1%, 2%, 3%, 4%, 5%, 6%, 7%, 8%, 9%, and 10%.

8. The method of claim 1, wherein the medication throughout a diagnostic taper sequence may be any medication that causes dependency, comprising methods of manufacture and dosage amounts varied to align with properties of the specific dependency-causing medication.

9. The method of claim 1, wherein dosages are formulated to deliver medication in various modalities, comprising:
a) tablets, films, adhesive patches, gels, liquids, or sprays;
b) creating the necessary dosages;
c) specifying amounts that are combined and mixed aligned with the properties of the modality of delivery.

10. The method of claim 1, wherein there are a range of embodiments, comprising:
a) each starting with an initial dosage that is the same every day for a fixed period of time, the starting dosage here called Baseline;
b) each initial dosage, i.e., each Baseline dosage, followed contiguously in time with a new total daily dosage level calculated based upon the Baseline dosage c) each new dosage called a “Step”;
d) commencing with a Step 1;
e) Step 1 followed by nine steps of equal duration of time, through Step 10;
e) the Baseline and the ten Steps being eleven distinct daily dosage levels;
f) each total daily dosage consisting of one dose or a number of divided doses, such as two divided doses or more, per day;
g) With a total daily starting dosage, i.e., Baseline, of 1, 2, 4 or 8 milligrams, or some other desired amount of buprenorphine;
h) the starting total daily dosage, “Baseline,” remaining at the same total dosage level each day for four weeks, and;
i) every new total daily dosage level, i.e., “Step” thereafter remaining at a constant total daily dosage for each new four-week period for every dosage level in the sequence of Baseline and ten Steps;
j) the rate of dosage reduction of the starting Baseline dosage being a one percent reduction from the Baseline dosage, that is, reducing from the last dosage of Baseline to the first dosage of Step 1, by one percent of the Baseline dosage;
k) maintaining that same total daily dosage level of the first total daily dosage of Step 1, throughout every day thereafter of Step 1, and;
I) at the first dose of every dosage level that follows, and consistently throughout at each dosage level, i.e., of Steps 2, 3, 4, 5, 6, 7, 8, 9, and 10, respectively, each said percentage reduction rate increasing;
m) at each successive Step the percent rate of reduction of the total daily dosage is one percent greater than the previous reduction, such that the reductions therefore are 2%, 3%, 4%, 5%, 6%, 7%, 8%, 9%, and 10% at the first dose of each successive Step respectively, and maintaining that dosage level throughout each dose of Steps 2, 3, 4, 5, 6, 7, 8, 9, and 10, respectively;
n) this arithmetic approach to dividing doses being applied to all dosing with divided doses, such that the divided doses on any given day, when added together, equal the total daily dosage for that day.

11. The method of claim 1, wherein the rates of dosage reduction from Baseline to the following Step or, from one Step to the next Step, are varied percentage amounts, comprising:
a) a starting dosage, i.e., Baseline, of 1, 2, 4 or 8 mg buprenorphine, and;
b) the time duration of the first dosage level, Baseline, and every dosage Step thereafter, being four weeks.

12. The method of claim 1, wherein the rates of dosage reduction from Baseline to the following Step or, from one Step to the next Step, are varied percentage amounts, comprising:
a) the rate of dosage reduction beginning with a 2% reduction from Baseline to Step 1, and, then;
b) said dosage reduction increases by 2% at each dosage reduction, such that the reductions are 4%, 6%, 8%, and 10% at the start of Steps 2, 3, 4, and 5, respectively, and;
c) said dosage reduction increases at the start of Step 6, a 10% reduction, with a 10% reduction at the start of Steps 7, 8, 9, and 10, respectively.

13. A method of formulating a diagnostic opioid taper regimen starting at a Baseline dosage with a sublingual tablet containing one milligram of buprenorphine, followed by Steps 1 through 10, in which Steps the rates of dosage reduction, effectuated at the start of each of these ten Steps, are 1%, 2%, 3%, 4%, 5%, 6%, 7%, 8%, 9%, and 10%, respectively, comprising:
a) employing a laboratory suitable for pharmaceutical formulation;
b) weighing 10.00 grams of buprenorphine;
c) placing the 10.00 grams of buprenorphine in an appropriate mixing vessel;
d) adding 990.00 grams of pharmaceutically appropriate excipients to the vessel;
e) mixing buprenorphine and excipients to total homogeneity, utilizing the methods necessary to achieve the desired homogeneity;
f) removing 100 mg aliquot of the powdered mixture;
g) compressing the 100 mg aliquot tablet that contains 1.00 (one) mg of buprenorphine, and setting the tablet aside for packaging;
h) repeating aliquot compression step to form tablets using applicable mass production protocols, when possible, until powder consumed, which process should make approximately 10,000 tablets;
i) commencing to make the second dosage in the sequence comprising a 100 mg gross weight tablet that contains 0.99 mg of buprenorphine;
j) placing 9.90 grams of buprenorphine in an appropriate mixing vessel;
k) adding 990.10 grams of pharmaceutically appropriate excipients,
l) mixing to total homogeneity;
m) removing 100 mg aliquot of the powdered mixture;
n) compressing the 100 mg gross weight aliquot tablet that contains 0.99 mg of buprenorphine;
o) repeating aliquot compression step, using applicable mass production protocols when possible, which process should make approximately 10,000 tablets;
p) repeating this process for the next nine dosage levels in this sequence, Steps 2 through 10, inclusive, such dosage levels being 0.97, 0.94, 0.90, 0.86, 0.81, 0.75, 0.69, 0.63, and, 0.57 milligrams of buprenorphine per 100 milligram tablet, as per above was done for the 1.00 mg and 0.99 mg of buprenorphine per 100 mg tablet, and following the same weighing and mixing and tablet compression protocols of buprenorphine and excipient selection for each dosage level;
q) making the said next nine steps, i.e, (0.97, 0.94; 0.90; 0.86; 0.81; 0.75; 0.69; 0.63, and; 0.57 milligrams of buprenorphine per 100 milligram tablet, by using the following quantities of (grams of buprenorphine per grams of excipient, that is, comprising mixtures of 9.70 grams buprenorphine per 990.30 grams of excipient; and the ratios of 9.41/990.59; 9.03/990.97; 8.58/991.42; 8.07/991.93; 7.50/992.50; 6.90/993.10; 6.28/993.72, and; 5.65 grams buprenorphine per 994.35 grams excipient;
r) adapting the approach, as needed, to divided doses;
s) calculating amounts and formulating active ingredient and excipients, by which approach the total daily dosages are divided into two or more doses per day;
t) packaging in such a manner as to make the manner of dispensing the regimen and administration, or self-administration, transparently clear to patient and provider as regards dosing sequences.

14. The method of claim 13, in which the medication is formulated into doses comprised of sublingual or buccal film, in which standardized methods of film formulation are employed to formulate dosages equal to total daily dosages, or any number of divided doses of the daily dosage, with a total daily dosages of, 1.00, 0.99, 0.97, 0.94; 0.90; 0.86; 0.81; 0.75; 0.69; 0.63, and; 0.57 milligrams or other range of doses in the same proportions, of buprenorphine or other selected medications.
